(19) United States
(12) Patent Application Publication
(10) Pub. No.: US 2023/0332239 A1
(43) Pub. Date: Oct. 19, 2023

(54) METHODS OF TREATING CANCER

(71) Applicant: ASTRAZENECA AB, Södertälje (SE)

(72) Inventors: Kris SACHSENMEIER, Wilmington, DE (US); Benjamin SIDDERS, Cambridge (GB); Robert D. MULLA, Wilmington, DE (US); Natalya MARKUZON, Wilmington, DE (US)

(21) Appl. No.: 17/756,426

(22) PCT Filed: Nov. 25, 2020
(86) PCT No.: PCT/EP2020/083397

§ 371 Date: May 25, 2022

Related U.S. Application Data
(60) Provisional application No. 62/940,329, filed on Nov. 26, 2019.

(51) Int. Cl. C12Q 1/6886

(52) U.S. Cl. CPC C12Q 1/6886; C12Q 2600/158; C12Q 2600/106; C12Q 2600/156

(57) ABSTRACT

Described herein are methods for identifying adenosine-driven cancers. The methods include determining a signature score of tumour adenosine signalling. The signature score reflects the expression levels of a signature group of genes whose pattern of expression levels is indicative of elevated adenosine signalling. Adenosine-driven cancers can be susceptible to treatment with an adenosine signalling inhibitor such as a CD39 inhibitor, a CD73 inhibitor, or an adenosine receptor antagonist. Methods of treating cancer are also described.

### CLAIM OF PRIORITY

This application claims priority to U.S. application No. 62/940,329, filed Nov. 26, 2019, the contents of which are incorporated by reference in their entirety.

### BACKGROUND

Immune checkpoint inhibitors hold great potential as cancer therapeutics. Nevertheless, clinical benefits from immune checkpoint inhibition have been modest. One potential explanation for the modest benefits is that tumours use nonoverlapping immunosuppressive mechanisms to facilitate immune escape.

Extracellular adenosine can suppress tumour infiltrating immune cells through a net negative impact of signalling through adenosine receptors, including the A2A receptor (A2AR). The primary source of extracellular adenosine in tumours is believed to be extracellular ATP, which is metabolized to AMP by the ectonucleotidase CD39, and then converted from AMP to adenosine by the ectonucleotidase CD73. Adenosine functions in processes such as cytoprotection, cell growth, angiogenesis and immunosuppression, and also plays a role in tumourigenesis.

### SUMMARY

In one aspect, a method for treating an adenosine-driven cancer in a subject includes diagnosing the subject with an adenosine-driven cancer. The subject can be diagnosed with an adenosine-driven cancer when, in a sample from the subject, a signature score of tumour adenosine signalling is greater than a predetermined cutoff value. The signature score can reflect the expression levels of a signature group of genes. The signature group of genes can include at least three genes selected from PPARG, CYBB, COL3A1, FOXP3, LAG3, APP, CD81, GPI, PTGS2, CASP1, FOS, MAPK1, MAPK3, CREB1, AKT3, TREM2, MUC1, CD164, FADD, FCGR2B, MASP2, ADA, SPA17, CCR5, CD55, IL17B, CD47, CCR2, CCL23, TARP, and EBI3. The method can include administering an effective amount of an adenosine signalling inhibitor to the diagnosed subject.

The signature score can be the GSVA score, mean, median, mode, or other statistical measure of the expression levels of the signature group of genes; and the signature score is optionally corrected for purity of the sample from the subject.

The predetermined cutoff value can be the median, mean, top quartile, top quintile, top decile, or other statistical measure, of the signature score in a selected group of reference samples, and wherein the signature score is optionally corrected for sample purity within the selected group of reference samples.

The selected group of reference samples can include a group of samples described in the Cancer Genome Atlas or a subset thereof.

In some embodiments, the signature score can be the GSVA score of the expression levels of the signature group of genes; wherein the predetermined cutoff value is the median GSVA score of the expression levels of the signature group of genes in a selected group of reference samples; wherein the selected group of reference samples includes a group of samples described in the Cancer Genome Atlas; and wherein the signature score for the selected group of reference samples is corrected for sample purity.

In some embodiments, the signature group of genes includes at least three genes selected from group A: PPARG, CYBB, COL3A1, FOXP3, LAG3, APP, CD81, GPI, PTGS2, CASP1, FOS, MAPK1, MAPK3, CREB1, AKT3, TREM2, MUC1, CD164, FADD, FCGR2B, MASP2, ADA, SPA17, CCR5, CD55, IL17B, CD47, CCR2, CCL23, TARP, and EBI3.

In some embodiments, the signature group of genes includes at least three genes selected from group B: PPARG, CYBB, COL3A1, FOXP3, LAG3, APP, CD81, GPI, PTGS2, CASP1, FOS, MAPK1, MAPK3, and CREB1.

In some embodiments, the signature group of genes includes at least three genes selected from group C: FOXP3, LAG3, CASP1, and CREB1.

In some embodiments, the signature group of genes includes at least three genes selected from group D: PTGS2, MAPK3, APP, MAPK1, FOS, and GPI.

In some embodiments, the signature group of genes includes at least three genes selected from group E: CYBB, LAG3, APP, CD81, GPI, PTGS2, CASP1, FOS, MAPK1, MAPK3, and CREB1.

In some embodiments, the signature group of genes includes at least three genes selected from group F: APP, FOS, CYBB, CREB1, AKT3, CD164, FADD, FCGR2B, ADA, CD47, and CCR2.

In some embodiments, the signature group of genes includes at least three genes selected from group G: PPARG, COL3A1, MAPK3, LAG3, CD81, APP, FOS, and CYBB.

In some embodiments, the signature group of genes includes at least three genes selected from group H: PPARG, COL3A1, MAPK3, LAG3, CD81, APP, FOS, CYBB, CASP1, TREM2, MUC1, MASP2, SPA17, CCR5, CD55, IL17B, CCL23, TARP, and EBI3.

In some embodiments, the signature group of genes includes at least three genes selected from group I: PTGS2, MAPK3, LAG3, CD81, APP, MAPK1, FOS, CYBB, CREB1, GPI, CASP1, CCR5, CD55, and TARP.

In some embodiments, wherein the signature group of genes includes:
at least five genes selected from group A;
at least five genes selected from group B;
at least three genes selected from group C;
at least five genes selected from group D;
at least five genes selected from group E;
at least five genes selected from group F;
at least five genes selected from group G;
at least five genes selected from group H; or
at least five genes selected from group I.

In some embodiments, the signature group of genes is:
group A;
group B;
group C;
group D;
group E;
group F;
group G;
group H; or
group I.

In some embodiments, the signature group of genes includes MAPK3, LAG3, CD81, APP, FOS, and CYBB.

Diagnosing the subject can further comprise determining that: the cancer has a mutation in one or more genes selected from VHL, ACVR2A, FIP1L1, NSD1, GATA3, or STK11.

Diagnosing the subject can further comprise determining that: the cancer has an SNV in one or more genes selected from MAML3, NPRL3, GATA3, BRD7, CISD2, KDM4E, KRT10, KRTAP5.5, NPEPPS, FIP1L1, KMT2B, RABL6, ITIH5, STK11, LOC100129697, PRDM9, UNC93B1, NSD1, HGC6.3, IRS1, VHL, ACVR2A, and MYO7A.

Diagnosing the subject can further comprise determining that: the cancer has a somatic copy number alteration (SCNA) at one or more locations selected from:
chr3 32098168:37495009,
chr3 1:17201156,
chr6 119669222:171115067,
chr19 39363864:39953130,
chr3 12384543:12494277,
chr19 30036025:30321189,
chr19 30183172:30321189,
chr1 1:29140747,
chr1 150637495:150740723,
chr1 228801039:249250621, and
chr8 113630879:139984811.

Diagnosing the subject can further comprise determining that the cancer has a mutation in a gene belonging to the TGF-β superfamily.

The cancer can be prostate cancer, breast cancer, colon cancer, lung cancer, uveal melanoma, cervical cancer, pancreatic cancer, or thyroid cancer.

In some embodiments, the cancer is prostate cancer. When the cancer is prostate cancer, the signature group of genes can include:
at least five genes selected from group E;
at least five genes selected from group F;
at least five genes selected from group G;
p at least five genes selected from group H; or
at least five genes selected from group I.

When the cancer is prostate cancer, the signature group of genes can include at least five genes selected from group I. When the cancer is prostate cancer, the signature group of genes can be group I.

The adenosine signalling inhibitor can include a CD39 inhibitor, a CD73 inhibitor, an adenosine receptor antagonist, or a combination thereof. The adenosine signalling inhibitor can be IPH5201, oleclumab, AZD4635, or a combination thereof.

The method can further include administering an effective amount of an immune checkpoint inhibitor to the diagnosed subject. The immune checkpoint inhibitor can be durvalumab, atezolizumab, avelumab, nivolumab, pembrolizumab, cemiplimab, tremelimumab, or ipilimumab.

In another aspect, use of an adenosine signalling inhibitor for the treatment of an adenosine-driven cancer in a subject, wherein: in a sample from the subject, a signature score of tumour adenosine signalling is greater than a predetermined cutoff value; wherein the signature score reflects the expression levels of a signature group of genes, wherein the signature group of genes includes at least three genes selected from group A, group B, group C, group D, group E, group F, group G, group H, and group I.

Other features, objects, and advantages will be apparent from the description and drawings, and from the claims.

### BRIEF DESCRIPTION OF THE DRAWINGS

FIG. 1A-1F: Signature validation. A) The adenosine signalling signature correlates (r²=0.92, p=0.018) with absolute adenosine levels in the tumour microenvironment in mouse syngeneic models. B) Effective A2AR inhibition, as defined by a reduced growth rate, with a specific small molecule inhibitor (AZD4635) in the MC38 syngeneic mouse model correlates with reduced adenosine signature scores (r²=−0.62, p=0.001). C) & D) The adenosine signature correlated with markers of NK cell exhaustion (r²=0.4, p<2.2e⁻¹⁶ and OR=3.1, p<2.2e⁻¹⁶) and CD8 T cell exhaustion (r²=0.6, p<2.2e⁻¹⁶ and OR=7.8, p<2.2e⁻¹⁶) in human tumours from TCGA. E) Adenosine signalling signature scores are reduced in A2AR KO CD11b+CD27−NK cells versus A2AR wild type NK cells from C57BL/6 mice. F) Adenosine signalling scores are reduced in 5 of 7 patients treated with AZD4635 in a Phase 1 trial, 4 of which have concomitant increases in gene expression signatures measuring cytolytic activity and IFNG signalling.

FIG. 2A-2D: Adenosine mediates survival in tumours of all types from TCGA. A) Overall survival is significantly worse (HR=0.6, Cox PH p<2.2e⁻¹⁶) in the upper quartile of all tumours from TCGA with the highest levels of adenosine signalling. B) The upper quartile also has a significantly worse progression free survival (HR=0.77, Cox PH p=0.0000006). C) Tumours with a high CD8 infiltrate (greater than the median level of CD8A expression) that are also adenosine high show an overall survival deficit (HR=0.47, Cox PH p<2.2e⁻¹⁶) compared to CD8 infiltrated tumours with low adenosine signalling. D) Likewise for progression free survival, tumours that are both CD8 infiltrated and adenosine high have a worse prognosis compared to those that are adenosine low (HR=0.65, Cox PH p=0.0000002).

FIG. 3A-3C: Adenosine signalling levels vary across tumour types. (A) Adenosine signalling across the tumour types of TCGA varies and is lowest in thymoma and highest in kidney renal clear cell carcinoma. B) Adenosine signalling association with overall survival in each tumour type from TCGA. C) Adenosine signalling association with progression free survival in each tumour type from TCGA. In B & C, boxes represent the hazard ratio (HR) when the upper quartile is compared to the lowest quartile, with whiskers describing the 95% confidence intervals.

FIG. 4A-4C: Genetic correlates of adenosine signalling. A) Adenosine signalling in pan-cancer disease segments defined by non-synonymous mutations at the gene level were compared to non-mutated samples. Circle size relates to number of mutated samples. Multiple testing corrected p-values (q) are shown versus the Cohen's D effect size where values >0 indicate higher levels in the mutant segment. B) As for A but each tumour type was studied independently. Circle size relates to number of mutated samples. C) Adenosine signalling in MSI versus MSS tumours from TCGA; MSI tumours have significantly higher levels of adenosine signalling.

FIG. 5A-5C: Adenosine signalling associates with TGF-β. A) Adenosine signalling levels are significantly higher in the TGF-β driven tumour cluster (C6) from Thorsson et al. B) Tumours from TCGA mutated in one of the 43 TGF-β superfamily members have higher levels of adenosine signalling versus TGF-β superfamily wild-type tumours. C) Tumours that are adenosine high and TGF-β superfamily mutant have worse overall survival compared to tumours that are adenosine low and TGF-β wild-type (HR=0.43, Cox PH p<2.2e⁻¹⁶), or those that are either TGF-β mutant (HR=0.74) or adenosine high (HR=0.72).

FIG. 6A-6C: Adenosine signalling is predictive for response to immunotherapy. A) Baseline tumour expression profiles from patients with a variety of solid tumours are higher in progressors versus responders to anti-PD1 therapy (either pembrolizumab or nivolumab) from Prat et al. (49) B) On treatment progression free survival is also significantly reduced in adenosine signalling high tumours (HR=0.29, Cox PH p=0.00012) but not in CD274 mRNA high tumours (HR=0.8, Cox PH p=0.47). Combining adenosine signature score and CD274 expression does not improve prognosis compared to the adenosine signature alone. C) Baseline tumour expression profiles from metastatic melanoma patients are higher in non-responders from Chen et al (50) despite only 6 genes from our 14 gene signature being present on the panel used.

FIG. 7: Adenosine signalling is confounded by tumour purity. The raw adenosine signalling score (A, C) is negatively correlated (r2=−0.44) with tumour purity (A. Blue line indicates a linear regression, red line a locally weighted regression (loess)). We correct for this using a linear model (B) and report purity adjusted adenosine signalling scores across cancer (D).

FIG. 8: the impact of adenosine signalling on immune cell levels. A) the pan-cancer spearman correlation of adenosine scores with immune cell content as determined by CIBERSORT. B) the spearman correlations of adenosine scores with immune cell content as determined by CIBERSORT for each individual tumour type.

FIG. 9: 6 adenosine associated genes have an established role in cancer pathogenesis, being members of the cancer gene census (39,40), including VHL, ACVR2A, FIP1L1 & NSD1 which all correlate with increased adenosine signalling, and GATA3 & STK11 that associate with reduced adenosine signalling.

FIG. 10: We found 55 SNVs associated with adenosine within an individual tumour type. 7 of these associations feature cancer census genes which are depicted here; TP53 in BRCA and STAD, GATA3 in BRCA, CDH1 in BRCA, VHL in KIRC, FIP1L1 in KIRP, STK11 in LUAD.

FIG. 11: Somatic copy number alterations (SCNA) are also associated with adenosine signalling. 124 SCNA are significant (q<0.05) with 11 having an effect size greater than 0.5. This includes a deletion on chromosome 3 which removes VHL and replicates the observation seen with SNVs.

FIG. 12: A) Adenosine signalling is a better predictor of PFS in response to anti-PD1 checkpoint therapy (data from Prat et al.) compared to B) CD274 mRNA expression. C) the combination of adenosine and CD274 expression does not outperform adenosine signalling alone.

FIG. 13: To further quantify adenosine signalling as a response predictor in the Prat et al and Chen at al cohorts of ICI treated patients, we used logistic regression to model the probability of a patient being a responder (CR, PR or SD) versus a non-responder (PD). The x-axis shows the adenosine signalling signature scores with scores for non-responders shown as dark blue dashes and responders as light blue dashes. The line describes the fitted model (with standard error) with the resulting probability of being a responder on the y-axis. A signature score just below 0 (−0.01368, red line) equates to a 50% probability of being a responder, and a signature score of −0.4 (purple line) equates to a 75% probability of being a responder. These figures need to be validated in much larger cohorts but indicate a possible route to a translatable cut point.

FIG. 14: A Kaplan-Meier curve showing progression free survival for a group of prostate cancer patients grouped as adenosine-high or adenosine-low, as determined by the signature of group I.

### DETAILED DESCRIPTION

Adenosine is a key suppressor of anti-cancer immune cell function and as such is a target of second-generation checkpoint inhibitors. There are several agents in early clinical trials targeting components of the adenosine pathway including A2AR, CD73, and CD39. Yet a need remains for the identification of cancers with a significant adenosine drive (and susceptibility to agents that target components of the adenosine pathway). However, it is challenging to measure tumour adenosine levels on a pan-cancer scale. A gene expression signature for adenosine signalling is described herein, and used to characterise the pan-cancer landscape of adenosine signalling and its role within the tumour microenvironment.

The role of the immune system in controlling cancer is widely recognized (1). Therapeutically, this is evidenced by a number of recent drug approvals for immunotherapy agents that enhance endogenous anti-tumour immunity (reviewed by (2)) or target tumours directly (reviewed by (3)). Responses to immunotherapies are distinct from those seen from other targeted therapies in at least two respects. Firstly, these responses are being observed in cancer indications of previously unmet need such as melanoma (4), lung adenocarcinoma (5) and haematological malignancies (6). Secondly, the duration of response to immunotherapy appears to persist for longer in certain settings than those observed with targeted therapies (reviewed by (7)).

The clinical success of immunotherapy has raised important questions regarding the initial or eventual failure to control disease, and the value of targeted vs. more integrated physiological approaches to tumour immunity. Current immunotherapies target specific molecules within the immune system, such as the checkpoint proteins PD1 and PDL1, and show responses in only a subset of cancer patients in any given indication. Total mutational burden (TMB) (8) and PDL1 protein (9) levels have been shown to correlate with immunotherapy response. However, only 30% of the responders are positive based upon these measures (10) suggesting that a more widespread response may be achieved by taking a broader approach; for example by targeting both innate as well as adaptive anti-tumour immunity.

One example of such a factor is the adenosine signalling axis (11), which has been shown to suppress NK and CD8+ T cell cytolytic activity whilst enhancing suppressive macrophage and dendritic cell polarisation as well as T-reg and MDSC proliferation (12). Beginning with landmark research by Sitkovsky (13), a series of preclinical studies (14-18) have been reported and clinical trials (19-21) initiated targeting adenosine signalling. Additionally, preclinical evidence supports a role for adenosine axis antagonists in chimeric antigen receptor T cell therapy (22), adoptive cell therapy (13) and cancer vaccines (23). Thus targeting the adenosine axis may block a broadly relevant immunosuppressive pathway in cancer (24).

It is therefore desirable to identify tumours where adenosine signalling is important to tumour survival and which may be susceptible to treatment by blockade of adenosine signalling.

Described herein are characteristics of the pan-cancer role of adenosine in human tumours, the relationship between adenosine signalling and prognosis of human tumours, and the identification of segments of disease where this relationship is more pronounced.

Adenosine signalling levels vary across the tumour types of TCGA, and this plays a central role in the suppression of anti-tumour immunity in tumours where an otherwise adequate CD8⁺ T cell infiltrate is present. Significant progress has been made in the identification of immune infiltrates alone that associate with outcomes (e.g. the Immunoscore (51)), yet orthogonal measures of immuno-suppressive effectors can provide additional information.

Genetic segments of disease that associate with higher adenosine signalling, including MSI tumours and specific genetic variation in TGF-β, are described herein. These mutations have potential as markers for adenosine-targeted therapies and are consistent with the concept that adenosine signalling acts to suppress the inflammatory response to highly immunogenic tumours (52). The relationship between adenosine signalling and TGF-β associates adenosine with fibroblast biology and reflects early clinical data from the anti-CD73 monoclonal antibody oleclumab in pancreatic cancer, an indication known to be rich in cancer-associated fibroblasts (53).

As used herein, the term “adenosine signalling signature” or “signature” refers to a pattern of gene expression that is characteristic of cellular response to adenosine signalling. The pattern of gene expression involves multiple genes whose expression is up- and down-regulated in a concordant manner when adenosine receptor signalling is present, e.g., signalling mediated by A2AR. In particular, the signature can be found in tumours which are undergoing adenosine signalling, i.e., a signature of tumour adenosine signalling. Those concordantly-regulated genes can be referred to collectively as a “signature group of genes”.

In some embodiments, the signature group of genes includes three or more genes selected from group A: PPARG, CYBB, COL3A1, FOXP3, LAG3, APP, CD81, GPI, PTGS2, CASP1, FOS, MAPK1, MAPK3, CREB1, AKT3, TREM2, MUC1, CD164, FADD, FCGR2B, MASP2, ADA, SPA17, CCR5, CD55, IL17B, CD47, CCR2, CCL23, TARP, and EBI3. In some embodiments, the signature includes five or more, seven or more, ten or more, fifteen or more, or twenty or more of group A.

In some embodiments, the signature group of genes includes three or more genes selected from group B: PPARG, CYBB, COL3A1, FOXP3, LAG3, APP, CD81, GPI, PTGS2, CASP1, FOS, MAPK1, MAPK3, and CREB1. In some embodiments, the signature includes five or more, seven or more, ten or more, twelve or more, or all of group B.

In some embodiments, the signature group of genes includes at least three genes selected from group C: FOXP3, LAG3, CASP1, and CREB1. In some embodiments, the signature group of genes is group C.

In some embodiments, the signature group of genes includes at least three genes selected from group D: PTGS2, MAPK3, APP, MAPK1, FOS, and GPI. In some embodiments, the signature group of genes is group D.

In some embodiments, the signature group of genes includes at least three genes selected from group E: CYBB, LAG3, APP, CD81, GPI, PTGS2, CASP1, FOS, MAPK1, MAPK3, and CREB1. In some embodiments, the signature includes five or more, or seven or more of group E. In some embodiments, the signature group of genes is group E.

In some embodiments, the signature group of genes includes at least three genes selected from group F: APP, FOS, CYBB, CREB1, AKT3, CD164, FADD, FCGR2B, ADA, CD47, and CCR2. In some embodiments, the signature includes five or more, or seven or more of group F. In some embodiments, the signature group of genes is group F.

In some embodiments, the signature group of genes includes at least three genes selected from group G: PPARG, COL3A1, MAPK3, LAG3, CD81, APP, FOS, and CYBB. In some embodiments, the signature includes five or more of group G. In some embodiments, the signature group of genes is group G.

In some embodiments, the signature group of genes includes at least three genes selected from group H: PPARG, COL3A1, MAPK3, LAG3, CD81, APP, FOS, CYBB, CASP1, TREM2, MUC1, MASP2, SPA17, CCR5, CD55, IL17B, CCL23, TARP, and EBI3. In some embodiments, the signature includes five or more, seven or more, ten or more, or fifteen or more of group H. In some embodiments, the signature group of genes is group H.

In some embodiments, the signature group of genes includes at least three genes selected from group I: PTGS2, MAPK3, LAG3, CD81, APP, MAPK1, FOS, CYBB, CREB1, GPI, CASP1, CCR5, CD55, and TARP. In some embodiments, the signature includes five or more, seven or more, or ten or more of group I. In some embodiments, the signature group of genes is group I.

As used herein, the term “signature score” refers to a quantitative measure of the signature, i.e., a numerical value indicative of the extent of adenosine signalling within a sample, e.g., a tumour sample. A signature score can correlate with intratumoural adenosine concentrations. Tumours can be classified according to a signature score as being candidates or non-candidates for treatment with one or more agents that suppress adenosine signalling.

A given sample (e.g., of tumour tissue) can be tested for expression levels of a signature group of genes and assigned a signature score based on the measured expression levels. Optionally, the signature score can reflect the expression levels of additional genes which are also indicative of adenosine signalling.

The signature score can be the GSVA score, mean, median, mode, or other statistical measure of the expression levels of the signature group of genes. Optionally, the signature score can be corrected for purity of the sample from the subject.

Adenosine mediates survival across tumours of all types and within specific indications, as described herein. Furthermore baseline adenosine signalling scores appear to predict response to immune checkpoint therapies, independently of PDL1 expression. In contrast adenosine signalling does not correlate with TMB. Because the signature has been derived independently of any specific molecular agent targeting the adenosine pathway, it may have utility across a broad spectrum of candidate drugs that target the adenosine pathway.

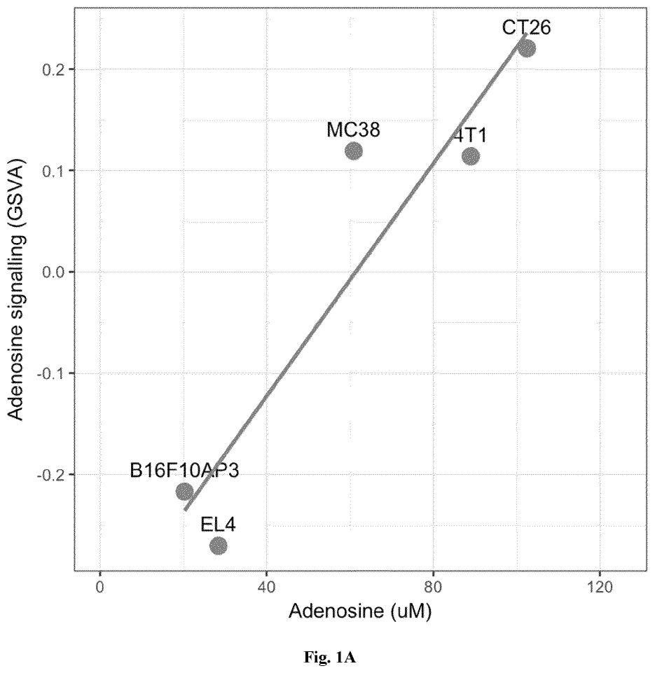

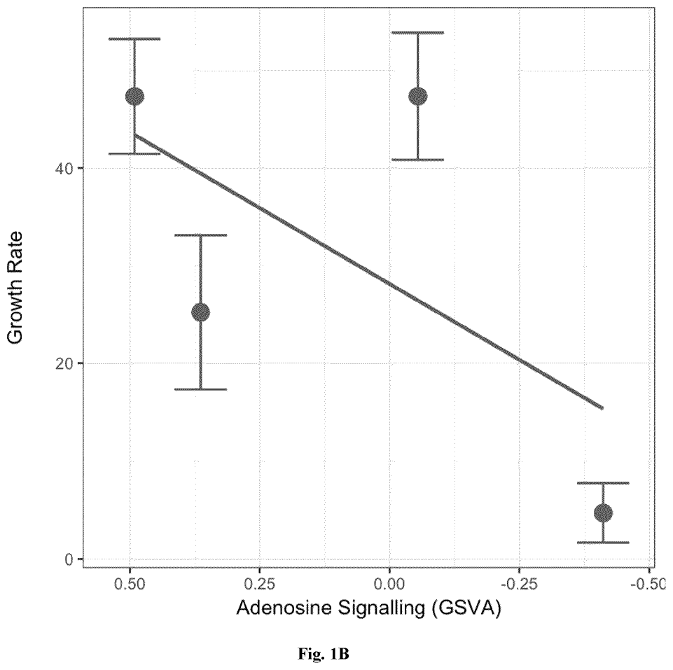

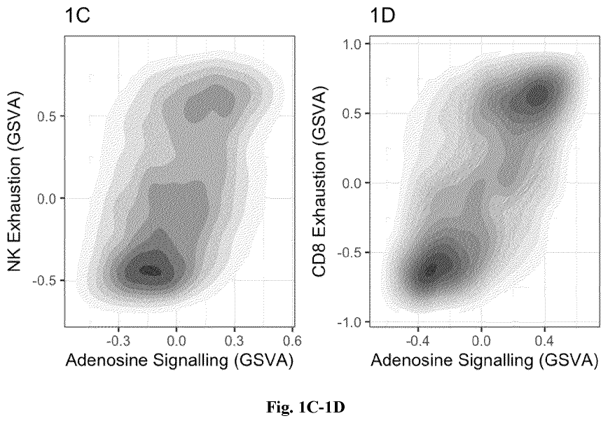

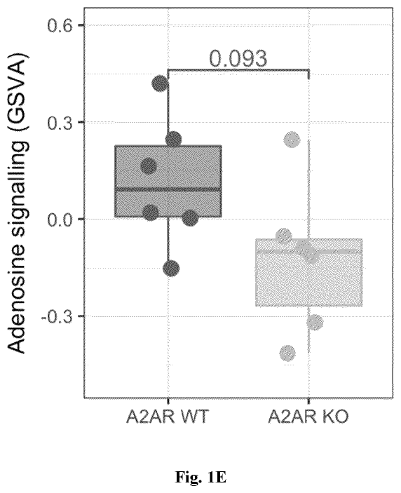

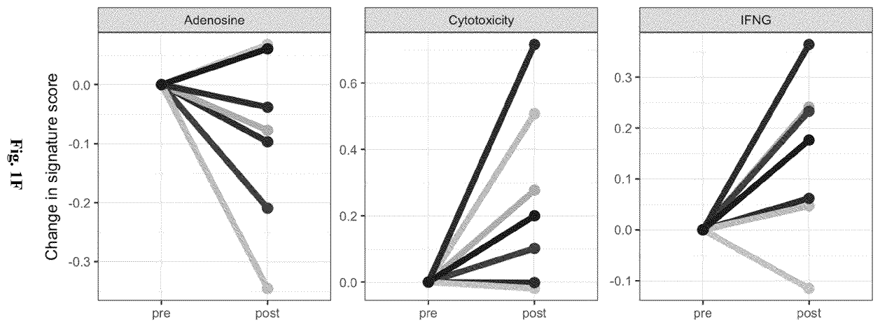

Described herein are several unexpected findings. Among them, the CT26 mouse model and MSI high human tumours were sensitive to immune checkpoint inhibitors yet we found both associated with high adenosine signalling (FIGS. 1A & 4C). In addition, not all tumour types with high adenosine signalling on average appeared to suffer a survival deficit. Further, although reduced adenosine signalling enriched for responders to checkpoint inhibition, not all adenosine low patients responded and vice versa. Immune checkpoint inhibitor sensitivity is likely determined by many factors in addition to adenosine. For example, the presence of CD8⁺ T cells, expression of PDL1 and high TMB are all associated with checkpoint response (54,55). It is also likely that an immune infiltration/response must occur prior to a state of adenosine mediated repression. As such, adenosine is another factor that contributes to the balance between those that induce antitumour immunity and those that are immuno-suppressive.

The signature described includes genes within a commercially available RNA expression panel, facilitating the translatability of the signature to clinical studies as well as direct comparison with other reported gene expression systems (56, 57). A group from Corvus Pharmaceuticals has taken an orthogonal approach to generating an adenosine related gene signature. Here, the authors identified genes up-regulated by NECA, an adenosine analogue, and suppressed by CPI-444, an A2AR antagonist. The two signatures have just one gene in common (PTGS2) which may reflect the compound specific nature of the CPI-444 signature. Expansion and further development of the signature described herein using a broader panel of transcripts could enhance the sensitivity of the signature.

Inflammatory signalling through ATP (58) or other nodes of the larger adenine nucleotide signalling axis (59) was not investigated here. A group from Corvus Pharmaceuticals has taken an orthogonal approach to generating an adenosine related gene signature. The authors identified genes up-regulated by NECA, an adenosine analogue, and suppressed by CPI-444, an A2AR antagonist (56). The two signatures have just one gene in common (PTGS2) which may reflect the compound-specific nature of the CPI-444 signature.

As used herein, the term “adenosine-driven cancer” refers to a cancer in which adenosine signalling pathways are more highly active than in other cancers. Adenosine-driven cancers can also be characterized by immune suppression in the tumour microenvironment due to adenosine signalling (e.g., adenosine signalling via A2AR, A2BR, or both). In other words, tumour growth may be driven by other factors than adenosine signalling, but adenosine signalling limits the degree to which the subject's immune response can attack the tumour. One way to identify an adenosine-driven cancer is by its adenosine signalling signature. In some embodiments, an adenosine-driven cancer can be an adenosine signalling inhibitor-sensitive cancer.

In some embodiments, a subject can be diagnosed with an adenosine-driven cancer if the signature score, in a sample from the subject, exceeds a predetermined cutoff value. The predetermined cutoff value can be assigned by first calculating the signature score for a set of reference samples (e.g., at least 25 samples, at least 50 samples, at least 100 samples, or more). The reference samples can be, for example, from different patients; and/or the same patients at different time points. The predetermined cutoff value can then be assigned after analysis of the signature scores of the reference samples. The predetermined cutoff value can be assigned as the median, mean, top quartile, top quintile, top decile, or other statistical measure of the signature scores of the reference samples. In some embodiments, the cutoff value is the median signature score of the reference samples. In some embodiments, the cutoff value can depend on the specific distributions the signature scores of the reference samples.

The set of reference samples can be from a group of patients having a variety of different cancers. Alternatively, the set of reference samples can be from a group of patients having a particular tumour type. In this context, a tumour type refers not only to the location of the cancer (e.g., prostate cancer or lung cancer), but can also refer to a narrower set of tumours, characterized by features such as tumour stage, mutation status of one or more genes, biomarker status, sensitivity to a given therapy, microsatellite instability, T-cell clonality, and others. Thus, even within a given type of cancer, sub-populations may be identified for which a different signature score is selected as the cutoff value. As one illustrative example, castration-resistant prostate cancer (CRPC) and castration-sensitive prostate cancer (CSPC), while both prostate cancers, can be considered different tumour types, as that term is used herein. Accordingly, the cutoff value can be different for different tumour types.

In some embodiments, the reference samples can be a group of samples described in The Cancer Genome Atlas (TCGA) or a subset thereof.

In analyzing the signature scores of the set of reference samples to predetermine a cutoff value, the signature scores of the reference samples can optionally be corrected for the purity of the individual samples, i.e., how much a given sample reflects expression levels of genes within tumour tissue as opposed to non-tumour tissue.

As used herein, the term “adenosine signalling inhibitor” refers to a compound (including without limitation small molecules and biologics) which interacts with one or more components of the adenosine signalling pathway in a manner capable of decreasing adenosine signalling. Thus, adenosine signalling inhibitors include, without limitation, compounds that inhibit the production of adenosine and compounds that antagonize one or more adenosine receptors. Thus, adenosine signalling inhibitors include compounds that inhibit enzyme(s) that directly or indirectly produce adenosine including, for example, CD39, CD73, and prostatic acid phosphatase (PAP). Examples of CD39 inhibitors include IPH5201 and POM-1. Examples of CD73 inhibitors include MEDI9447 (oleclumab) and AB680. Adenosine signalling inhibitors also include compounds that antagonize one or more adenosine receptors (including, for example, A1R, A2AR, A2BR and A3R).

An adenosine-driven cancer can, in some embodiments, be an adenosine receptor antagonist-sensitive cancer. As used herein, “an adenosine receptor antagonist-sensitive cancer” refers to a cancer that responds to treatment with an adenosine receptor antagonist (whether alone or in combination with another treatment). The adenosine receptor antagonist can be an antagonist of one or more of the A1R, A2AR, A2BR, and A3R adenosine receptors. Examples of adenosine receptor antagonists include without limitation AZD4635 (chemical name: 6-(2-chloro-6-methylpyridin-4-yl)-5-(4-fluorophenyl)-1,2,4-triazin-3-amine), CPI-444, PBF-509, PBF-1129, and preladenant.

Antibodies and antibody-like compounds (e.g., monoclonal antibodies, antibody fragments, and the like) that bind to CD39, CD73, PAP, or an adenosine receptor can also be adenosine signalling inhibitors. Adenosine signalling inhibitors can also include compounds that inhibit downstream components of the adenosine signalling pathway.

Administration of one or more adenosine signalling inhibitors to a subject diagnosed with an adenosine-driven cancer can promote a positive therapeutic response with respect to the adenosine-driven cancer. As used herein, the term “positive therapeutic response,” encompasses a reduction or inhibition of the progression and/or duration of cancer, the reduction or amelioration of the severity of cancer, and/or the amelioration of one or more symptoms thereof. For example, a reduction or inhibition of the progression and/or duration of cancer can be characterized as a complete response. The term “complete response” refers to an absence of clinically detectable disease with normalization of any previously abnormal test results. Alternatively, an improvement in the disease can be categorized as being a partial response.

In some illustrative examples, a positive therapeutic response includes one, two or three or more of the following results: (1) a stabilization, reduction or elimination of the cancer cell population; (2) a stabilization or reduction in cancer growth; (3) an impairment in the formation of cancer; (4) eradication, removal, or control of primary, regional and/or metastatic cancer; (5) an increase in anti-cancer immune response; (6) a reduction in mortality; (7) an increase in disease-free, relapse-free, progression-free, and/or overall survival, duration, or rate; (8) an increase in the response rate, the durability of response, or number of patients who respond or are in remission; (9) a decrease in hospitalization rate, (10) a decrease in hospitalization lengths, (11) the size of the cancer is maintained and does not increase or increases by less than 10%, preferably less than 5%, preferably less than 4%, preferably less than 2%, (12) an increase in the number of patients in remission, and (13) a decrease in the number or intensity of adjuvant therapies (e.g., chemotherapy or hormonal therapy) that would otherwise be required to treat the cancer.

In some embodiments, certain markers can supplement the signature as a way to identify adenosine-driven cancers. Such markers can include high microsatellite instability (or “MSI-high”) status; mutations in genes such as VHL, ACVR2A, FIP1L1, NSD1, GATA3 and STK11; single nucleotide variations (described in more detail below); and mutations in genes belonging to the TGF-beta superfamily (also described in more detail below).

Described herein are methods for treating an adenosine-driven cancer in a subject. The methods can include diagnosing the subject with an adenosine-driven cancer. Diagnosing the subject with an adenosine-driven cancer can include determining the subject's adenosine signature score. The signature score can be compared to a predetermined cutoff value to identify subjects having, or not having, an adenosine-driven cancer.

Determining the subject's adenosine signature score can include measuring, in a sample from the subject, the expression levels of a signature group of genes. In other words, changes in the expression levels of one or more genes in the signature is representative of changes in the degree, extent or intensity of signalling via the adenosine pathway.

The degree, extent or intensity of signalling via the adenosine pathway can refer to one or more properties including: concentrations of adenosine precursors (ATP, ADP, AMP) in the tumour microenvironment; concentrations and/or activity levels of enzymes that are involved in the conversion of adenosine precusors to adenosine (e.g., CD39, CD73, and PAP); whether the enzymes are cell-surface bound or soluble; concentration of adenosine in the tumour microenvironment; degree or extent of adenosine receptor occupancy (including A1, A2A, A2B, and A3, particularly A2A and A2B receptors, more particularly A2A receptor); level of intracellular G-protein activity mediated by A1, A2A, A2B, and A3, particularly A2A and A2B receptors; and the degree, extent or intensity of effects that occur in the adenosine signalling pathway downstream of the adenosine receptor.

The signature group of genes can include at least three genes selected from PPARG, CYBB, COL3A1, FOXP3, LAG3, APP, CD81, GPI, PTGS2, CASP1, FOS, MAPK1, MAPK3, and CREB1. In some embodiments, the signature group of genes includes at least five genes; at least seven genes; at least ten genes; or at least 12 genes selected from PPARG, CYBB, COL3A1, FOXP3, LAG3, APP, CD81, GPI, PTGS2, CASP1, FOS, MAPK1, MAPK3, and CREB1. In some embodiments, the signature group of genes includes all of PPARG, CYBB, COL3A1, FOXP3, LAG3, APP, CD81, GPI, PTGS2, CASP1, FOS, MAPK1, MAPK3, and CREB1, and optionally one or more additional genes. In some embodiments, the signature group of genes includes only PPARG, CYBB, COL3A1, FOXP3, LAG3, APP, CD81, GPI, PTGS2, CASP1, FOS, MAPK1, MAPK3, and CREB1.

In some embodiments, the adenosine-driven cancer can be uveal melanoma, cervical cancer, pancreatic cancer, thyroid cancer, prostate cancer, lung cancer, bladder cancer, or other cancer. In some embodiments, the elevated adenosine cancer can be prostate cancer.

In some embodiments, the sample is a tumour sample (e.g., a biopsy sample), a circulating tumour DNA (ctDNA) sample, a plasma RNA sample, an exosome sample, or other blood-derived sample. The expression levels of the signature group of genes can be measured by any method that can quantify mRNA levels in a sample from a subject, particularly a sample that reflects mRNA levels as expressed in tumour cells. Suitable methods for measuring expression levels include, but are not limited to, RNAseq, qPCR, or platform-specific assays such as microarrays or nanostring analysis.

Methods of treating an adenosine-driven cancer in a subject can include administering an effective amount of an adenosine signalling inhibitor to a subject diagnosed with an adenosine-driven cancer.

In some embodiments, the methods of treating further include administering an effective amount of an immune checkpoint inhibitor to the diagnosed subject. The immune checkpoint inhibitor can be, for example, durvalumab, atezolizumab, avelumab, nivolumab, pembrolizumab, cemiplimab, tremelimumab, or ipilimumab.

In one aspect, a method for treating an adenosine-driven cancer in a subject can include: diagnosing the subject with an adenosine-driven cancer when, in a sample from the subject, a signature score of tumour adenosine signalling is greater than a predetermined cutoff value; wherein the signature score is the GSVA score of at least three genes selected from one of group A, group B, group C, group D, group E, group F, group G, group H, and group I; and administering an effective amount of an adenosine signalling inhibitor to the diagnosed subject. In some embodiments, the signature score is the mean, median, mode, or other statistical measure of the expression levels of at least three genes selected from one of group A, group B, group C, group D, group E, group F, group G, group H, and group I.

In one aspect, a method for treating an adenosine-driven cancer in a subject can include: measuring, in a sample from the subject, a signature score of tumour adenosine signalling that is greater than a predetermined cutoff value; wherein the signature score reflects the expression levels of a signature group of genes, wherein the signature group of genes includes at least three genes selected from one of group A, group B, group C, group D, group E, group F, group G, group H, and group I; and administering an effective amount of an adenosine signalling inhibitor to the diagnosed subject.

In one aspect, a method for treating an adenosine-driven cancer in a subject can include: obtaining a sample from the subject; measuring, in the sample from the subject, a signature score of tumour adenosine signalling, wherein the signature score is greater than a predetermined cutoff value; wherein the signature score reflects the expression levels of a signature group of genes, wherein the signature group of genes includes at least three genes selected from one of group A, group B, group C, group D, group E, group F, group G, group H, and group I; and administering an effective amount of an adenosine signalling inhibitor to the diagnosed subject.

In one aspect, a method for treating an adenosine-driven cancer in a subject can include: identifying a subject having a value of a signature score that is greater than a predetermined cutoff value; wherein the signature score reflects the expression levels of a signature group of genes, wherein the signature group of genes includes at least three genes selected from one of group A, group B, group C, group D, group E, group F, group G, group H, and group I; and administering an effective amount of an adenosine signalling inhibitor to the diagnosed subject.

In one aspect, a method of identifying a subject having a cancer suited to treatment with an adenosine signalling inhibitor can include: determining that a signature score of tumour adenosine signalling is greater than a predetermined cutoff value in a sample from a subject; wherein the signature score reflects the expression levels of a signature group of genes, wherein the signature group of genes includes at least three genes selected from one of group A, group B, group C, group D, group E, group F, group G, group H, and group I.

In one aspect, a method of identifying an adenosine-driven cancer in a subject can include: determining a signature score of tumour adenosine signalling in a sample from the subject; wherein the signature score reflects the expression levels of a signature group of genes, wherein the signature group of genes includes at least three genes selected from one of group A, group B, group C, group D, group E, group F, group G, group H, and group I; and determining whether the signature score is greater than a predetermined cutoff value.

In one aspect, a method of treating an adenosine-driven cancer in a subject can include: determining a signature score of tumour adenosine signalling in a sample from a subject; determining whether the signature score is greater than a predetermined cutoff value; and administering an effective amount of an adenosine signalling inhibitor to the subject.

In one aspect, an adenosine signalling inhibitor can be for use in the treatment of cancer (e.g., an adenosine-driven cancer) in a subject in need thereof, wherein: in a sample from the subject, a signature score of tumour adenosine signalling is greater than a predetermined cutoff value.

In one aspect, a method of predicting a subject's response to a cancer treatment (e.g., a treatment for an adenosine-driven cancer) can include: comparing a signature score of tumour adenosine signalling in a sample from the subject to predetermined cutoff value, wherein the signature score reflects the expression levels of a signature group of genes, wherein the signature group of genes includes at least three genes selected from one of group A, group B, group C, group D, group E, group F, group G, group H, and group I.

In one aspect, a method of diminishing adenosine-mediated immunosuppression in a tumour of a subject can include: determining whether, in a sample from the subject, a signature score of tumour adenosine signalling is greater than a predetermined cutoff value; and administering an effective amount of an adenosine signalling inhibitor to the subject if the signature score is greater than the predetermined cutoff value.

### REFERENCES

Each of the references herein are incorporated by reference in their entirety.

1. Schreiber R D, Old L J, Smyth M J. Cancer Immunoediting: Integrating Immunity's Roles in Cancer Suppression and Promotion [Internet]. Science. 2011. page 1565-70. Available from: http://dx.doi.org/10.1126/science.1203486

2. Serrano P, Hartmann M, Schmitt E, Franco P, Amexis G, Gross J, et al. Clinical Development and Initial Approval of Novel Immune Checkpoint Inhibitors in Oncology: Insights From a Global Regulatory Perspective. Clin Pharmacol Ther. 2019; 105:582-97.

3. June C H, O'Connor R S, Kawalekar O U, Ghassemi S, Milone M C. CAR T cell immunotherapy for human cancer [Internet]. Science. 2018. page 1361-5. Available from: http://dx.doi.org/10.1126/science.aar6711

4. Boyle G M. Therapy for metastatic melanoma: an overview and update. Expert Rev Anticancer Ther. 2011; 11:725-37.

5. Gandhi L, Rodriguez-Abreu D, Gadgeel S, Esteban E, Felip E, De Angelis F, et al. Pembrolizumab plus Chemotherapy in Metastatic Non-Small-Cell Lung Cancer. N Engl J Med. 2018; 378:2078-92.

6. Landoni E, Savoldo B. Treating hematological malignancies with cell therapy: where are we now? Expert Opin Biol Ther. 2018; 18:65-75.

7. Wargo J A, Cooper Z A, Flaherty K T. Universes Collide: Combining Immunotherapy with Targeted Therapy for Cancer [Internet]. Cancer Discovery. 2014. page 1377-86. Available from: http://dx.doi.org/10.1158/2159-8290.cd-14-0477

8. Rizvi N A, Hellmann M D, Snyder A, Kvistborg P, Makarov V, Havel J J, et al. Mutational landscape determines sensitivity to PD-1 blockade in non-small cell lung cancer [Internet]. Science. 2015. page 124-8. Available from: http://dx.doi.org/10.1126/science.aaa1348

9. Reck M, Rodriguez-Abreu D, Robinson A G, Hui R, Csőszi T, Fülöp A, et al. Pembrolizumab versus Chemotherapy for PD-L1—Positive Non-Small-Cell Lung Cancer [Internet]. New England Journal of Medicine. 2016. page 1823-33. Available from: http://dx.doi.org/10.1056/nejmoa1606774

10. Hellmann M D, Nathanson T, Rizvi H, Creelan B C, Sanchez-Vega F, Ahuja A, et al. Genomic Features of Response to Combination Immunotherapy in Patients with Advanced Non-Small-Cell Lung Cancer [Internet]. Cancer Cell. 2018. page 843-52.e4. Available from: http://dx.doi.org/10.1016/j.ccell.2018.03.018

11. Vijayan D, Young A, Teng M W L, Smyth M J. Targeting immunosuppressive adenosine in cancer. Nat Rev Cancer. 2017; 17:709-24.

12. Young A, Mittal D, Stagg J, Smyth M J. Targeting cancer-derived adenosine: new therapeutic approaches. Cancer Discov. 2014; 4:879-88.

13. Ohta A, Gorelik E, Prasad S J, Ronchese F, Lukashev D, Wong M K K, et al. A2A adenosine receptor protects tumours from antitumour T cells. Proc Natl Acad Sci USA. National Academy of Sciences; 2006; 103:13132.

14. Willingham S B, Ho P Y, Hotson A, Hill C, Piccione E C, Hsieh J, et al. A2AR Antagonism with CPI-444 Induces Antitumour Responses and Augments Efficacy to Anti-PD-(L)1 and Anti-CTLA-4 in Preclinical Models. Cancer Immunol Res. 2018; 6:1136-49.

15. Borodovsky A, Wang Y, Ye M, Shaw J C, Sachsenmeier K F, Deng N, et al. Abstract 5580: Preclinical pharmacodynamics and antitumour activity of AZD4635, a novel adenosine 2A receptor inhibitor that reverses adenosine mediated T cell suppression. Cancer Res. 2017; 77:5580-5580.

16. Pinna A. Adenosine A2A receptor antagonists in Parkinson's disease: progress in clinical trials from the newly approved istradefylline to drugs in early development and those already discontinued. CNS Drugs. 2014; 28:455-74.

17. Houthuys E, Marillier R, Deregnaucourt T, Brouwer M, Basilico P, Pirson R, et al. Abstract LB-291: EOS100850, an insurmountable and non-brain penetrant A2Areceptor antagonist, inhibits adenosine-mediated T cell suppression, demonstrates anti-tumour activity and exhibits best-in class characteristics [Internet]. Cancer Research. 2018. page LB-291. Available from: http://dx.doi.org/10.1158/1538-7445.am2018-lb-291

18. Galezowski M, Wegrzyn P, Bobowska A, Commandeur C, Dziedzic K, Nowogrodzki M, et al. Abstract 3770: Characterization of novel dual A2A/A2Badenosine receptor antagonists for cancer immunotherapy [Internet]. Cancer Research. 2018. page 3770-3770. Available from: http://dx.doi.org/10.1158/1538-7445.am2018-3770

19. Emens L, Powderly J, Fong L, Brody J, Forde P, Hellmann M, et al. Abstract CT119: CPI-444, an oral adenosine A2A receptor (A2AR) antagonist, demonstrates clinical activity in patients with advanced solid tumours [Internet]. Cancer Research. 2017. page CT119-CT119. Available from: http://dx.doi.org/10.1158/1538-7445.am2017-ct119

20. Chiappori A, Williams C C, Creelan B C, Tanvetyanon T, Gray J E, Haura E B, et al. Phase I/II study of the A2AR antagonist NIR178 (PBF-509), an oral immunotherapy, in patients (pts) with advanced NSCLC [Internet]. Journal of Clinical Oncology. 2018. page 9089-9089. Available from: http://dx.doi.org/10.1200/jco.2018.36.15_suppl.9089

21. Research C M, Case Medical Research. First-in-Human Study of EOS100850 in Patients With Cancer [Internet]. Case Medical Research. 2019. Available from: http://dx.doi.org/10.31525/ct1-nct03873883

22. Beavis P A, Henderson M A, Giuffrida L, Mills J K, Sek K, Cross R S, et al. Targeting the adenosine 2A receptor enhances chimeric antigen receptor T cell efficacy. J Clin Invest. 2017; 127:929-41.

23. Arab S, Kheshtchin N, Ajami M, Ashurpoor M, Safvati A, Namdar A, et al. Increased efficacy of a dendritic cell-based therapeutic cancer vaccine with adenosine receptor antagonist and CD73 inhibitor. Tumour Biol. 2017; 39:1010428317695021.

24. Leone R D, Emens L A. Targeting adenosine for cancer immunotherapy [Internet]. Journal for ImmunoTherapy of Cancer. 2018. Available from: http://dx.doi.org/10.1186/s40425-018-0360-8

25. Chindelevitch L, Ziemek D, Enayetallah A, Randhawa R, Sidders B, Brockel C, et al. Causal reasoning on biological networks: interpreting transcriptional changes. Bioinformatics. 2012; 28:1114-21.

26. Fakhry C T, Choudhary P, Gutteridge A, Sidders B, Chen P, Ziemek D, et al. Interpreting transcriptional changes using causal graphs: new methods and their practical utility on public networks. BMC Bioinformatics. 2016; 17:318.

27. Sidders B, Brockel C, Gutteridge A, Harland L, Jansen P G, McEwen R, et al. Precompetitive activity to address the biological data needs of drug discovery. Nat Rev Drug Discov. 2014; 13:83-4.

28. Jamieson D G, Roberts P M, Robertson D L, Sidders B, Nenadic G. Cataloging the biomedical world of pain through semi-automated curation of molecular interactions. Database. 2013; 2013:bat033.

29. Jamieson D G, Moss A, Kennedy M, Jones S, Nenadic G, Robertson D L, et al. The pain interactome: connecting pain-specific protein interactions. Pain. 2014; 155:2243-52.

30. Biorelate—Curating Truths in Biomedicine [Internet]. [cited 2019 May 15]. Available from: https://www.biorelate.com/

31. Ingenuity Pathway Analysis—QIAGEN Bioinformatics [Internet]. QIAGEN Bioinformatics. [cited 2019 Mar. 5]. Available from: https://www.qiagenbioinformatics.com/?qia-storyline=products/ingenuity-pathway-analysis

32. Sheth S, Brito R, Mukherjea D, Rybak L, Ramkumar V. Adenosine Receptors: Expression, Function and Regulation [Internet]. International Journal of Molecular Sciences. 2014. page 2024-52. Available from: http://dx.doi.org/10.3390/ijms15022024

33. Mosely S I S, Prime J E, Sainson R C A, Koopmann J-O, Wang D Y Q, Greenawalt D M, et al. Rational Selection of Syngeneic Preclinical Tumour Models for Immunotherapeutic Drug Discovery. Cancer Immunology Research. 2016; 5:29-41.

34. Borodovsky A, Wang Y, Ye M, Shaw J C, Sachsenmeier K, Deng N, et al. Abstract 3751: Inhibition of A2AR by AZD4635 induces anti-tumour immunity alone and in combination with anti-PD-L1 in preclinical models. Cancer Res. 2018; 78:3751-3751.

35. Young A, Ngiow S F, Gao Y, Patch A-M, Barkauskas D S, Messaoudene M, et al. A2AR Adenosine Signalling Suppresses Natural Killer Cell Maturation in the Tumour Microenvironment. Cancer Res. 2018; 78:1003-16.

36. Newman A M, Liu CL, Green M R, Gentles A J, Feng W, Xu Y, et al. Robust enumeration of cell subsets from tissue expression profiles. Nat Methods. 2015; 12:453-7.

37. Vallon V, Mühlbauer B, Osswald H. Adenosine and kidney function. Physiol Rev. 2006; 86:901-40.

38. Köröskényi K, Joós G, Szondy Z. Adenosine in the Thymus. Front Pharmacol. 2017; 8:932.

39. Futreal P A, Andrew Futreal P, Coin L, Marshall M, Down T, Hubbard T, et al. A census of human cancer genes. Nat Rev Cancer. 2004; 4:177-83.

40. Forbes S A, Beare D, Boutselakis H, Bamford S, Bindal N, Tate J, et al. COSMIC: somatic cancer genetics at high-resolution. Nucleic Acids Res. 2017; 45:D777-83.

41. Hatfield S M, Kjaergaard J, Lukashev D, Belikoff B, Schreiber T H, Sethumadhavan S, et al. Systemic oxygenation weakens the hypoxia and hypoxia inducible factor 1α-dependent and extracellular adenosine-mediated tumour protection. J Mol Med. 2014; 92:1283-92.

42. Ahmad S F, Ansari M A, Nadeem A, Bakheet S A, Almutairi M M, Attia S M. Adenosine A2A receptor signalling affects IL-21/IL-22 cytokines and GATA3/T-bet transcription factor expression in CD4 T cells from a BTBR T Itpr3tf/J mouse model of autism. J Neuroimmunol. 2017; 311:59-67.

43. Skoulidis F, Goldberg M E, Greenawalt D M, Hellmann M D, Awad M M, Gainor J F, et al. Mutations and PD-1 Inhibitor Resistance in-Mutant Lung Adenocarcinoma. Cancer Discov. 2018; 8:822-35.

44. Shaw R J, Bardeesy N, Manning B D, Lopez L, Kosmatka M, DePinho R A, et al. The LKB1 tumour suppressor negatively regulates mTOR signalling. Cancer Cell. 2004; 6:91-9.

45. Shen K, Huang R K, Brignole E J, Condon K J, Valenstein M L, Chantranupong L, et al. Architecture of the human GATOR1 and GATOR1-Rag GTPases complexes. Nature. 2018; 556:64-9.

46. Yang L, Pang Y, Moses H L. TGF-β and immune cells: an important regulatory axis in the tumour microenvironment and progression. Trends Immunol. 2010; 31:220-7.

47. Thorsson V, Gibbs D L, Brown S D, Wolf D, Bortone D S, Ou Yang T-H, et al. The Immune Landscape of Cancer. Immunity. 2018; 48:812-30.e14.

48. Korkut A, Zaidi S, Kanchi R S, Rao S, Gough N R, Schultz A, et al. A Pan-Cancer Analysis Reveals High-Frequency Genetic Alterations in Mediators of Signalling by the TGF-β Superfamily. Cell Syst. 2018; 7:422-37.e7.

49. Prat A, Navarro A, Paré L, Reguart N, Galván P, Pascual T, et al. Immune-Related Gene Expression Profiling After PD-1 Blockade in Non-Small Cell Lung Carcinoma, Head and Neck Squamous Cell Carcinoma, and Melanoma. Cancer Res. 2017; 77:3540-50.

50. Chen P-L, Roh W, Reuben A, Cooper Z A, Spencer C N, Prieto P A, et al. Analysis of Immune Signatures in Longitudinal Tumour Samples Yields Insight into Biomarkers of Response and Mechanisms of Resistance to Immune Checkpoint Blockade. Cancer Discov. 2016; 6:827-37.

51. Pagès F, Mlecnik B, Marliot F, Bindea G, Ou F-S, Bifulco C, et al. International validation of the consensus Immunoscore for the classification of colon cancer: a prognostic and accuracy study. Lancet. 2018; 391:2128-39.

52. Kjaergaard J, Hatfield S, Jones G, Ohta A, Sitkovsky M. A Adenosine Receptor Gene Deletion or Synthetic A Antagonist Liberate Tumour-Reactive CD8 T Cells from Tumour-Induced Immunosuppression. J Immunol. 2018; 201:782-91.

53. Ahrens D von, von Ahrens D, Bhagat T D, Nagrath D, Maitra A, Verma A. The role of stromal cancer-associated fibroblasts in pancreatic cancer [Internet]. Journal of Hematology & Oncology. 2017. Available from: http://dx.doi.org/10.1186/s13045-017-0448-5

54. Gibney G T, Weiner L M, Atkins M B. Predictive biomarkers for checkpoint inhibitor-based immunotherapy. Lancet Oncol. 2016; 17:e542-51.

55. Samstein R M, Lee C-H, Shoushtari A N, Hellmann M D, Shen R, Janjigian Y Y, et al. Tumour mutational load predicts survival after immunotherapy across multiple cancer types. Nat Genet. 2019; 51:202-6.

56. Willingham S, Hotson A N, Laport G, Kwei L, Fong L, Sznol M, et al. 1137PDIdentification of adenosine pathway genes associated with response to therapy with the adenosine receptor antagonist CPI-444 [Internet]. Annals of Oncology. 2018. Available from: http://dx.doi.org/10.1093/annonc/mdy288.010

57. Kim E, Palackdharry S, Yaniv B, Mark J, Tang A, Wilson K, et al. Gene expression signature after one dose of neoadjuvant pembrolizumab associated with tumour response in head and neck squamous cell carcinoma (HNSCC) [Internet]. Journal of Clinical Oncology. 2018. page 6059-6059. Available from: http://dx.doi.org/10.1200/jco.2018.36.15_suppl.6059

58. Draganov D, Gopalakrishna-Pillai S, Chen Y-R, Zuckerman N, Moeller S, Wang C, et al. Modulation of P2X4/P2X7/Pannexin-1 sensitivity to extracellular ATP via Ivermectin induces a non-apoptotic and inflammatory form of cancer cell death. Sci Rep. 2015; 5:16222.

59. Fliegert R, Heeren J, Koch-Nolte F, Nikolaev V O, Lohr C, Meier C, et al. Adenine nucleotides as paracrine mediators and intracellular second messengers in immunity and inflammation. Biochem Soc Trans. 2019; 47:329-37.

60. Hänzelmann S, Castelo R, Guinney J. GSVA: gene set variation analysis for microarray and RNA-seq data. BMC Bioinformatics. 2013; 14:7.

61. Lai Z, Markovets A, Ahdesmaki M, Chapman B, Hofmann O, McEwen R, et al. VarDict: a novel and versatile variant caller for next-generation sequencing in cancer research. Nucleic Acids Res. 2016; 44:e108.

62. Hoadley K A, Yau C, Hinoue T, Wolf D M, Lazar A J, Drill E, et al. Cell-of-Origin Patterns Dominate the Molecular Classification of 10,000 Tumours from 33 Types of Cancer. Cell. 2018; 173:291-304.e6.

63. Liu J, Lichtenberg T, Hoadley K A, Poisson L M, Lazar A J, Cherniack A D, et al. An Integrated TCGA Pan-Cancer Clinical Data Resource to Drive High-Quality Survival Outcome Analytics. Cell. 2018; 173:400-16.e11.

64. Sanchez-Vega F, Mina M, Armenia J, Chatila W K, Luna A, La K C, et al. Oncogenic Signalling Pathways in The Cancer Genome Atlas. Cell. 2018; 173:321-37.e10.

65. Taylor A M, Shih J, Ha G, Gao G F, Zhang X, Berger A C, et al. Genomic and Functional Approaches to Understanding Cancer Aneuploidy. Cancer Cell. 2018; 33:676-89.e3.

66. Kim D, Langmead B, Salzberg S L. a fast spliced aligner with low memory requirements. Nat Methods. 2015; 12:357-60.

67. Patro R, Duggal G, Love M I, Irizarry R A, Kingsford C. Salmon provides fast and bias-aware quantification of transcript expression. Nat Methods. 2017; 14:417-9.

68. Therneau T M. Survival Analysis [R package survival version 2.44-1.1]. Comprehensive R Archive Network (CRAN); [cited 2019 May 15]; Available from: https://CRAN.R-project.org/package=survival

69. Goodwin K J, Gangl E, Sarkar U, Pop-Damkov P, Jones N, Borodovsky A, et al. Development of a quantification method for adenosine in tumours by LC-MS/MS with dansyl chloride derivatization. Anal Biochem. 2019; 568:78-88.

### EXAMPLES

The following examples are illustrative and not intended to be limiting. Other embodiments are within the scope of the following claims.

### Methods

### Signature Generation & Scoring

To define a network of regulatory interactions for the A2AR receptor we used two complementary datasets. Natural Language Processing of abstracts and open-access full-text from Medline and PubMed Central was performed as previously described in (28) by Biorelate® Ltd (30) to broadly sweep as much of the literature as possible. In contrast, knowledge derived purely from manual curation in the Ingenuity Pathway Analysis tool database (Qiagen) were used to provide a deeper mining of full text articles from a smaller set of high-impact journals targeted by that resource.

Biorelate® define a causal (regulatory) interaction as a relationship between two entities (genes or proteins) where the subject (cause) entity has a directed edge with an object (theme) entity. Gene entity terms and their relationships from their in-house dictionaries were matched through their machine-learning named-entity-recognition software, now incorporated within Biorelate Galactic AI™. Protein entities from human, mouse and rat were retained under the expectation that human data would be the most relevant, whilst mouse and rat would capture the majority of animal models used in biomedical research. Causal interactions were then collapsed such that all events containing the same pair of entities and the same interaction type were grouped. These groups were assigned a confidence score that was used to rank select events for manual verification.

We then filter the combined set of regulatory relationships to identify genes that are downstream of A2AR (154 genes; 136 from manual curation and 18 from NLP, with 13 detected in both), up-regulated by A2AR (90 genes; 78 from manual curation and 12 from NLP), robustly expressed in human tumours, defined as having a median expression greater than the median expression of all genes (74 genes; 66 from manual curation and 8 from NLP), and, finally, by their presence on the Nanostring PanCancer Immune Profiling expression panel (14 genes; 10 from manual curation and 4 from NLP). This last step ensures that our signature retains maximum clinical utility given that the Nanostring panel is widely used to profile FFPE samples from clinical trials where whole transcriptome profiling is often unavailable. The 14 genes that meet these criteria and form the signature are: PPARG, CYBB, COL3A1, FOXP3, LAG3, APP, CD81, GPI, PTGS2, CASP1, FOS, MAPK1, MAPK3, CREB1.

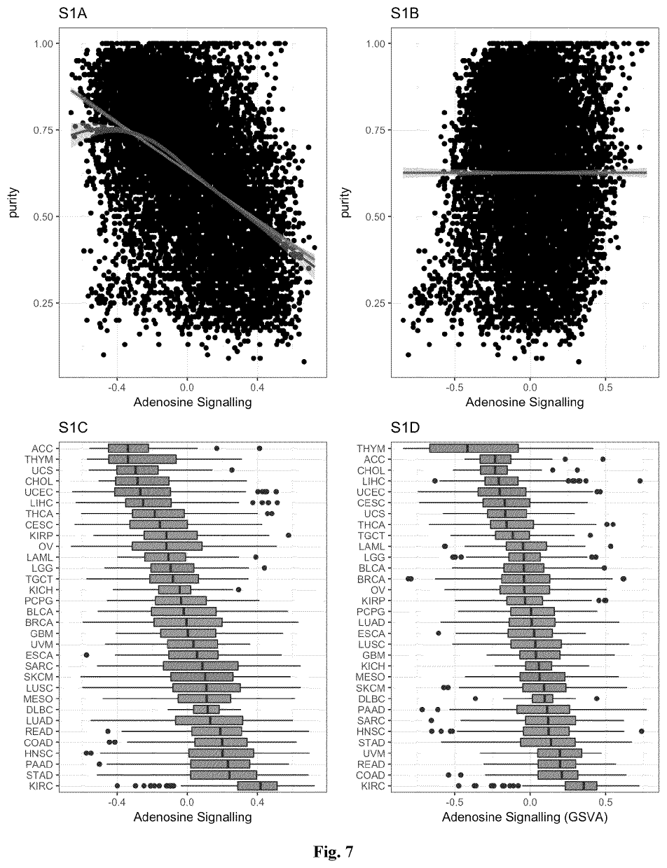

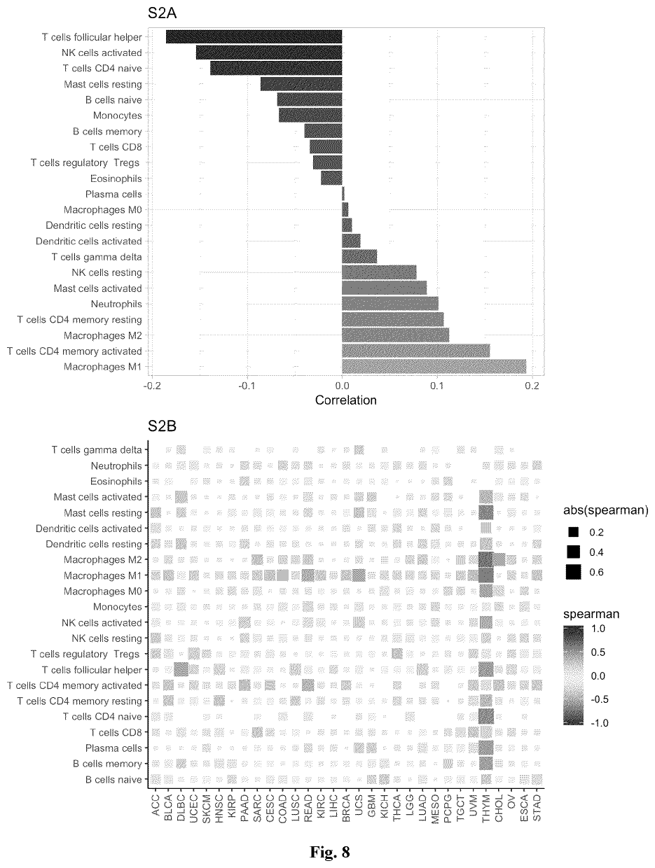

We scored transcriptome data with the signature using GSVA (60). This method is robust to outlying genes expressed at different orders of magnitude and generates scores amenable to downstream statistical interpretation. We observe a strong linear correlation between signatures representing immune processes/features and tumour purity in TCGA (see FIG. 7). To account for this bias we adjust the signature scores for tumour purity by fitting a linear regression derived from all samples in TCGA versus tumour purity and then applying the following correction:

corrected score=uncorrected score−(intercept+slope*purity)

### Analysis of Public Datasets

Exome sequencing data from TCGA were processed as described in (61). TCGA RNAseq data were described in (62) and associated clinical data were taken from (63). Copy number variants made with GISTIC version 2.0.22 were obtained from the TCGA Firehose. MSI subtype information were obtained from (64). Tumour purity data were obtained from (65).

RNAseq data from ADORA2A knock-out NK cell lines generated in (35) were obtained from the European Nucleotide Archive (PRJEB22631). Reads were aligned to the mouse genome (mm10) using HISAT2 (66) and expression levels were quantified using Salmon (67).

Published cohorts of immuno-therapy treated subjects with pre-processed gene expression profiles were obtained from (49,50) and scored with GSVA. The anti-CTLA4 dataset (50) was generated with a custom nanostring panel that contained only 6 (CASP1, CD81, CYBB, LAG3, PARG, PTGS2) of the 14 genes from our signature.

### Survival Analysis

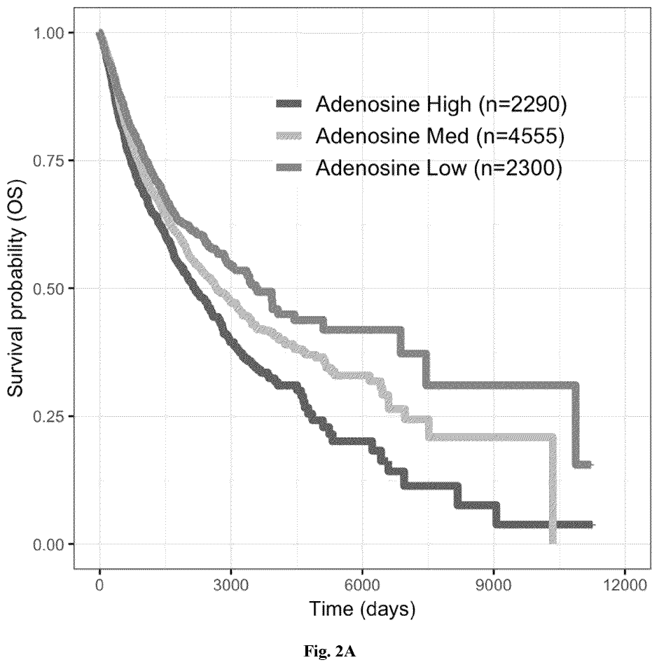

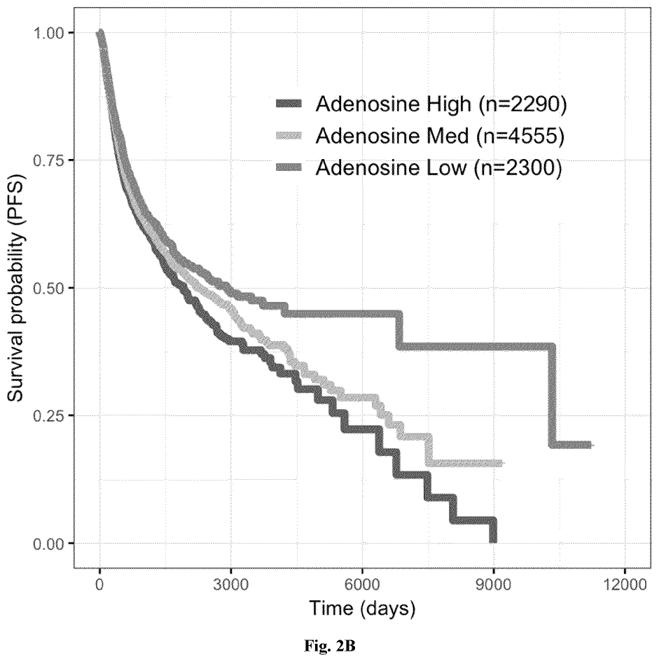

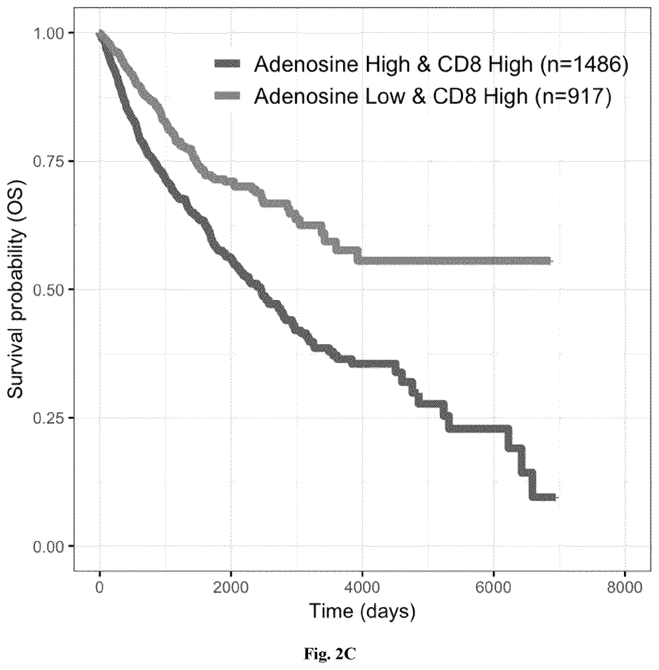

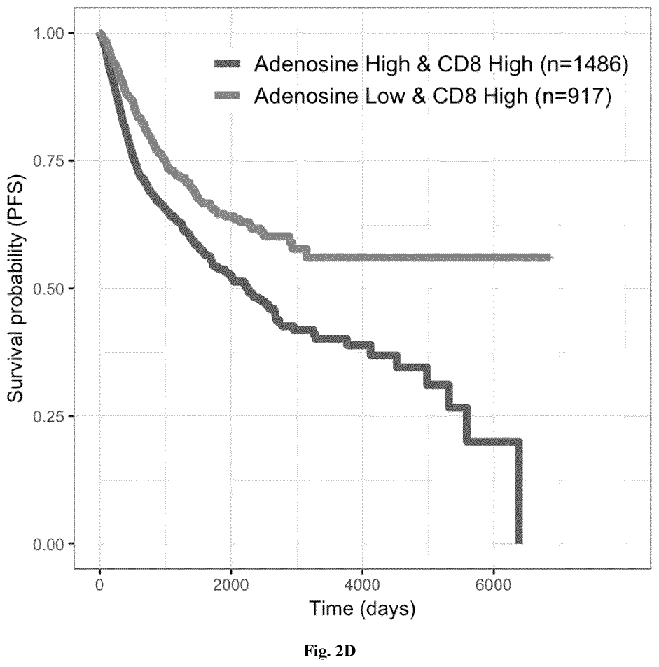

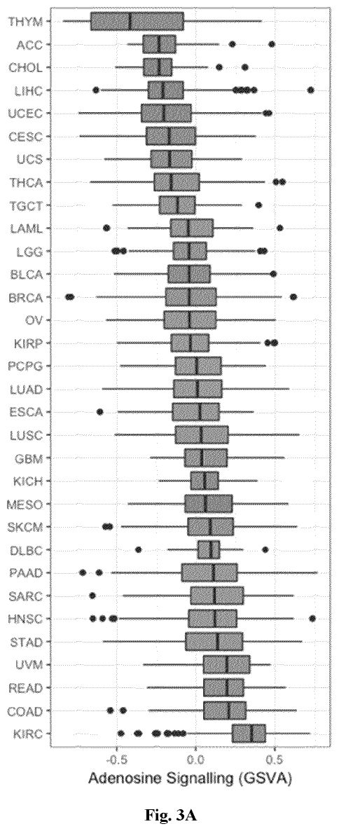

Survival analyses were performed using the Cox Proportional Hazards regression model as implemented in the Survival package from R (68). For the analysis presented in FIGS. 2A & B tumours were split into high (>75th), medium (25-75th) and low (<25th) based on quartiles. In all other survival analysis adenosine signature scores were split on 0 with >0 high and <0 low.

### Immune Cell-Type Infiltrate Scoring

Immune cell infiltrates were determined with an SVR approach based on CIBERSORT (36) to define relative immune cell abundance. To study the association of adenosine with CD8⁺ T cell infiltration we consider CD8 high tumours to be greater than the median of CD8A expression across all samples. All other cell or cell-state signatures were scored using GSVA. NK cell exhaustion was determined using expression of KIR3DL1, KIR3DL2, IL2RA, IL15RA, HAVCR2 and EOMES. Cytotoxicity was determined using the expression of: NKG7, CST7, PRF1, GZMA, GZMB and IFNG. CD8 Exhaustion was determined using the signature provided in (Danaher et al. 2017). IFNG signalling was determined using the signature presented by (Ayers et al. 2017).

### Genetic Associations with Adenosine

Genetic associations with adenosine signalling were studied for all genes with a mutation frequency >2% across the cohort being studied and for all copy number variants. A linear model with tumour type, TMB and MSI status as covariates was fit to the data and ANOVA was used to test for significance. Effect sizes were computed as the Cohen's D effect size where the difference between means is normalised for the variance within the data. All p values were adjusted for multiple testing using the Benjamini-Hochberg procedure.

### Mouse Models for Signature Validation

All animal studies were performed according to AstraZeneca Institutional Animal Care and Use Committee guidelines.

Transcriptional profiling data for the 5 syngeneic models shown in FIG. 1A were obtained from (33). Tumour adenosine measurements from syngeneic models were performed as described in Goodwin et al (69).

For the in vivo treatment study shown in FIG. 1B, MC38 cells were confirmed free of mycoplasma and mouse pathogens by PCR as part of a rodent pathogen testing panel (IMPACT, IDEXX Bioresearch). Thawed cells were cultured in DMEM supplemented with 10% heat-inactivated FBS and 1% L-glutamine (Sigma Aldrich) at 37° C. in a humidified incubator maintained at 5% CO2. Cell counts were performed prior to implantation by Countess Cell Counter (Invitrogen). For subcutaneous implants, 5×10−5 MC38 cells/mouse were re-suspended in sterile PBS and injected subcutaneously into the right flanks of 4-6 week old female C57BL/6 mice (Charles River Labs) in a total volume of 0.1 ml/mouse.

Mice were randomized into treatment groups at a starting tumour volume of 50-90 mm3. AZD4635 nanosuspension formulation (Aptuit, Verona) was reconstituted in sterile water and dosed orally twice daily (BID) at 50 mg/kg. Tumour volume and body weight were measured twice weekly after randomization. Growth rate was calculated as the slope of a linear model fit to the percent change in tumour volume from day 0 over time.

### Human Phase 1A Study of AZD4635

The first-in-human trial, NCT02740985, was conducted to assess safety, PK and pharmacodynamic activity of AZD4635 as monotherapy and in combination with durvalumab in patients with treatment refractory solid tumours. Pre-dose and on-treatment tumour biopsies were collected from 7 subjects who were treated with AZD4635 monotherapy at or below the maximum tolerated dose (MTD) of 100 mg PO daily.

Total RNA was extracted from tumour tissue macrodissected from 5 mm thick FFPE sections using the miRNeasy FFPE Kit (QIAGEN). RNA integrity and quantity were assessed on the TapeStation 2200 using the RNA ScreenTape System (Agilent). Manufacturer's recommended protocols were followed.

The RNA was subsequently analyzed for gene expression using the NanoString nCounter FLEX Analysis System and the commercially available 770-gene, human PanCancer Immune Profiling Panel (NanoString). Following the manufacturer's standard XT CodeSet Gene Expression Assays protocol, 25-100 ng RNA was hybridized with Capture and Reporter probes at 65° C. for 22 hours. Post-hybridization sample processing on the Prep Station using the high sensitivity setting was followed by data collection on the Digital Analyzer scanning at 555 fields of view (FOV). Pre-processing of the raw count data, which included background subtraction of the negative control probes, positive control normalization and housekeeping gene normalization, was performed in the nSolver 4.0 (NanoString) software using the geometric means and default parameters. All samples included in downstream analyses fell within the default nSolver QC parameters.

### Example 1: A Gene Expression Signature Accurately Captures Adenosine Signalling Levels

It is challenging to measure tumour adenosine levels in a high-throughput manner, so we sought to create a gene expression signature that would recapitulate adenosine signalling and allow us to study the wealth of existing data from large collections of tumour transcriptomes. It has previously been shown that causal, or regulatory, protein/gene interaction knowledge is a powerful substrate for the interpretation of transcriptomic data (25-27). We thus sought to compile a regulatory network for the adenosine signalling pathway. Both Natural Language Processing (NLP), as described previously (28-30), and manually extracted knowledge (31) were used to define a network of interactions between the A2AR receptor and downstream entities. Of the four adenosine receptors, A2A was selected as the basis of our study given that A1 and A3 function to increase cAMP rather than decrease it, which is necessary for immune cell suppression (32). A2B has considerably lower affinity for adenosine (32). Thus A2A gives us the cleanest signal with which to capture the immuno-suppressive effects of adenosine. We focused on regulatory interactions where there was evidence that A2AR increased expression of the downstream entity in the primary scientific literature. We found 172 genes that have been reported to be regulated by A2AR, 90 of which were reported to be positively regulated by A2AR signalling activity. We applied additional filters to ensure the genes are robustly expressed in human tumours and present on a widely used clinical transcriptomics assay. Our final signature consisted of 14 genes whose concordant activity is indicative of adenosine signalling; PPARG, CYBB, COL3A1, FOXP3, LAGS, APP, CD81, GPI, PTGS2, CASP1, FOS, MAPK1, MAPK3, CREB1.

To confirm the validity and specificity of our signature we quantified the intra-tumoural levels of adenosine in five murine syngeneic models for which we also have transcriptional profiles (33). We find a significant correlation (r²=0.92, p=0.018) between measured intra-tumour adenosine concentrations and adenosine signalling as captured using our signature (FIG. 1A). We next assessed whether the adenosine signature tracked with inhibition of the adenosine receptor in vivo within the MC38 syngeneic model using AZD4635, an A2AR selective small molecule currently in clinical development (15,34). We find that our signature correlates (r²=−0.62, p=0.001) with reduced growth rate after A2AR inhibition (FIG. 1B). Furthermore, knock-out of the A2AR receptor (35) abrogated adenosine signalling signature scores in CD11b+CD27−NK cells (FIG. 1E). A key biological effect of adenosine within human tumours is to suppress immune cell activity (35). In concordance with this, the adenosine signature scores have a significant association with NK cell (r²=0.4, p<2.2e⁻¹⁶ and OR=3.1, p<2.2e⁻¹⁶, FIG. 1C) and CD8⁺ T cell (r²=0.6, p<2.2e⁻¹⁶ and OR=7.8, p<2.2e⁻¹⁶, FIG. 1D) exhaustion marker expression in TCGA. Finally, seven patients with a variety of solid tumours were treated once daily with AZD4635 in a Phase 1A study (NCT02740985) to assess pharmacodynamic changes in signature scores within humans. Adenosine signalling scores were reduced in 5 of the 7 (70%) patients, 4 of which also had concordant increases in gene expression signatures of cytolytic activity and IFNG signalling. Taken together these data demonstrate that our proposed signature is a useful surrogate for adenosine signalling activity when studying bulk transcriptomes of human and mouse tumours.

### Example 2: Adenosine Mediates Survival in Human Disease

Having established that our signature captures adenosine signalling activity within tumours, we next explored the role of adenosine signalling in dictating disease outcomes. Adenosine suppresses a functional anti-tumour response and so we would expect tumours with a high adenosine drive to be more aggressive and have reduced survival. To confirm this we used our signature scores to compare survival in tumours with high adenosine signalling to tumours with low adenosine signalling across all cancers in TCGA. However, before doing so we studied the potential for tumour purity to bias our scores across large datasets. We observed that low purity trended with greater adenosine signature scores (FIG. 7A). We therefore established a normalisation of signature scores for tumour purity (FIG. 7B & methods) to remove this bias from further studies of human tumours in TCGA.

Adenosine signalling high tumours were defined as the upper quartile of signature scores across all samples, and likewise adenosine low consisted of the lower quartile. We find that high levels of adenosine signalling associate with significantly worse overall survival (HR=0.6, Cox PH p<2.2e⁻¹⁶) and progression free survival (HR=0.77, Cox PH p=0.0000006) in a pan-cancer model (FIGS. 2A & B). This association remains if the data are split by tertiles (OS HR=0.75, Cox PH p=0.000000006; PFS HR=0.83, Cox PH p=0.000025) or on the median (OS HR=0.81, Cox PH p=0.0000002; PFS HR=0.86, Cox PH p=0.00007).

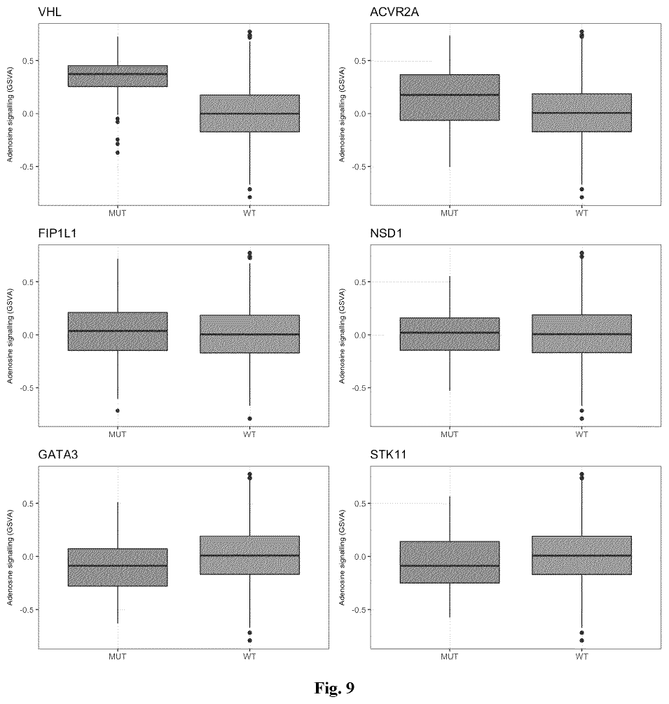

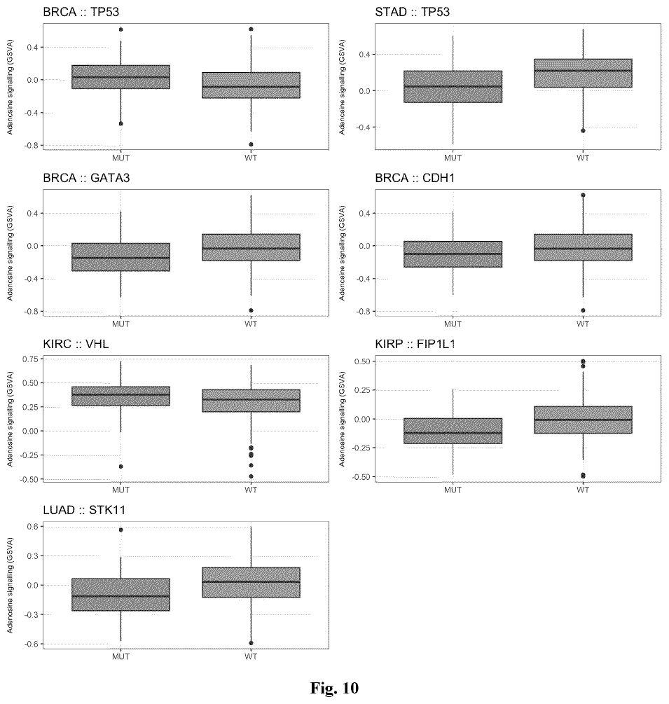

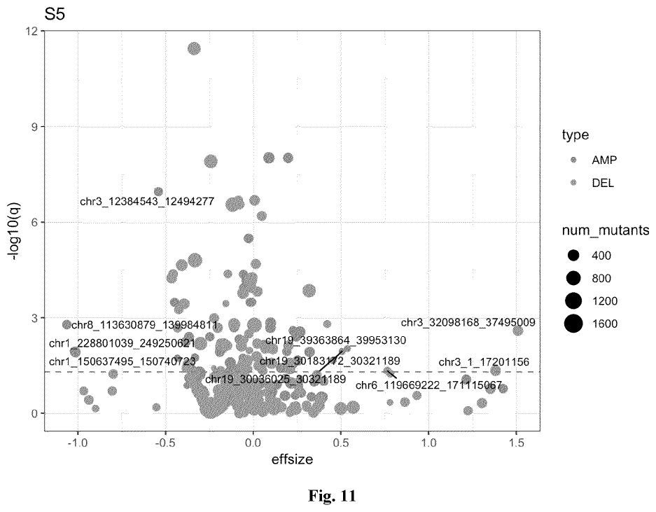

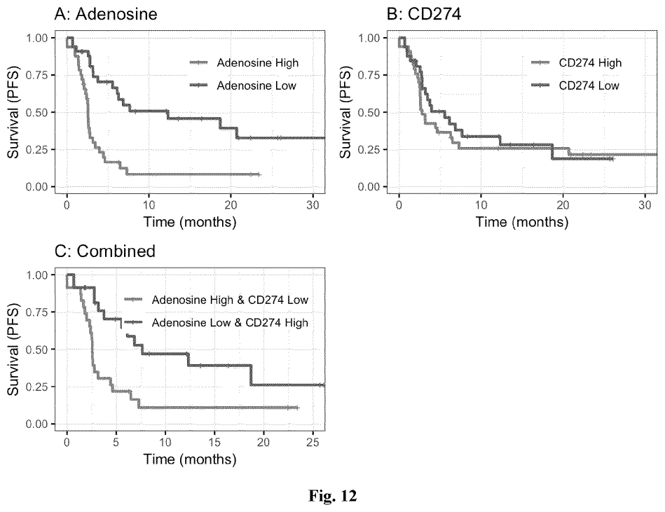

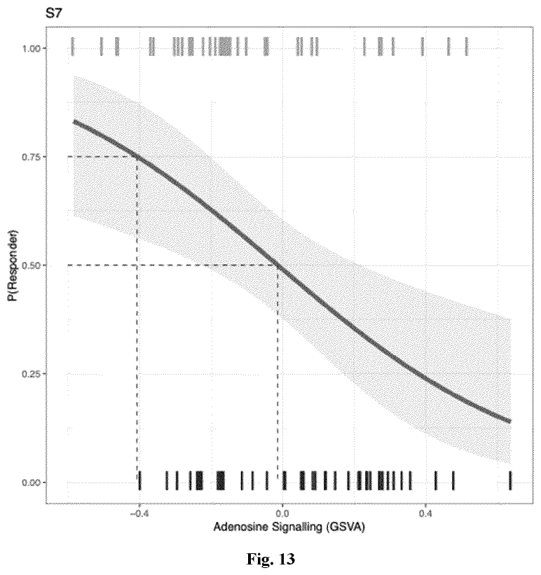

Considerable progress has been made in the characterisation of the tumour microenvironment from the perspective of immune cell infiltration. However, it remains unclear why some apparently ‘hot’ tumours with an otherwise adequate infiltration of immune cells do not appear to mount an effective antitumour response. We first assessed the relationship of adenosine signalling to immune cell infiltrates inferred from bulk RNAseq in TCGA using a support vector regression approach based upon the CIBERSORT algorithm (36). There are no strong associations but we observe weak negative correlations with activated NK cell & T follicular helper cell scores and a positive correlation with resting NK cell and macrophage scores (FIG. 9A). We therefore studied the ability of adenosine to modulate the activity of existing immune infiltrates by studying only tumours with a high level of CD8⁺ T cell infiltration, defined as greater than the median of CD8A expression across all samples. We find a dramatic survival deficit in tumours that are both CD8 high and adenosine high versus tumours that are CD8 high but adenosine low, for both overall survival (HR=0.47, Cox PH p<2.2e⁻¹⁶) and progression free survival (HR=0.65, Cox PH p=0.0000002) (FIGS. 2C & 2D). Further, the survival deficit between adenosine high and low tumours is reduced or ablated in CD8 low tumours (OS Cox PH p=0.001, PFS Cox PH p=0.05).

### Example 3: Adenosine Signalling in Individual Tumour Types

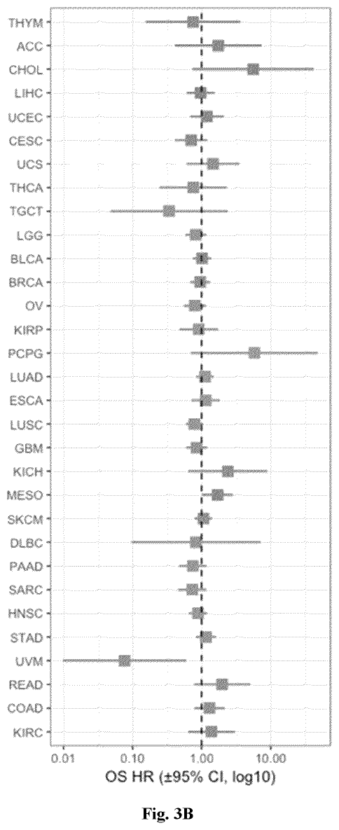

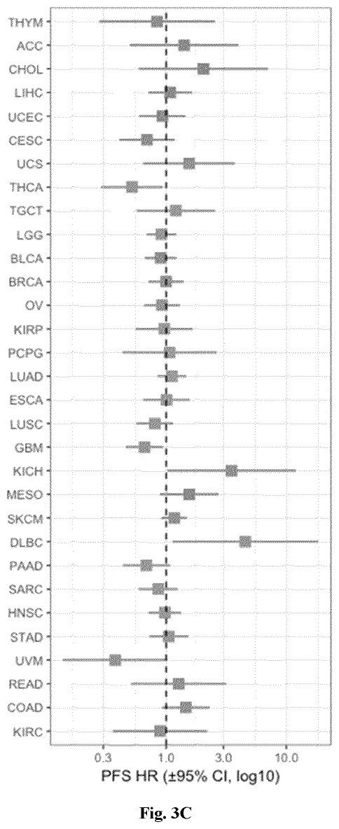

We next studied the adenosine signalling profile of each tumour type from TCGA individually. All tumour types exhibit a wide range of adenosine signalling levels and all have some individuals with high adenosine signalling (FIG. 3A). Kidney renal clear cell carcinoma (KIRC) has the highest levels of adenosine signalling on average across all tumour types whereas thymoma (THYM) has the lowest (FIG. 3A). Consistent with this observation, adenosine is known to play an important role within the kidney where it regulates a variety of physiological functions and is present at significant extracellular concentrations (37). Interestingly adenosine also plays a role in the thymus, regulating the thymocyte selection process (38).

Concordantly reduced overall and progression free survival in adenosine high tumours is seen in 13 individual diseases (FIG. 3C), with four having an HR <0.7 for both survival measures; uveal melanoma (UVM, OS HR=0.08, PFS HR=0.38), cervical (CESC, OS HR=0.70, PFS HR=0.69), pancreatic (PAAD, OS HR=0.74, PFS HR=0.68) and thyroid (THCA, OS HR=0.75, PFS HR=0.52). However, uveal melanoma (UVM, HR=0.08, p=0.016) is the only case where OS is statistically significant for an individual tumour type (FIG. 3B). Similarly, glioblastoma (GBM, HR=0.66, p=0.02), thyroid carcinoma (THCA, HR=0.52, p=0.03) and uveal melanoma (UVM, HR=0.37, p=0.05) are the only diseases where adenosine signalling is statistically associated with worse progression free survival. Interestingly, DLBCL appears to derive a progression free survival benefit from high levels of adenosine signalling (DLBC, HR=5.19, p=0.02) although the data is highly variable and notably is not concordant with overall survival.

### Example 4: Genetic Correlates of Adenosine Signalling

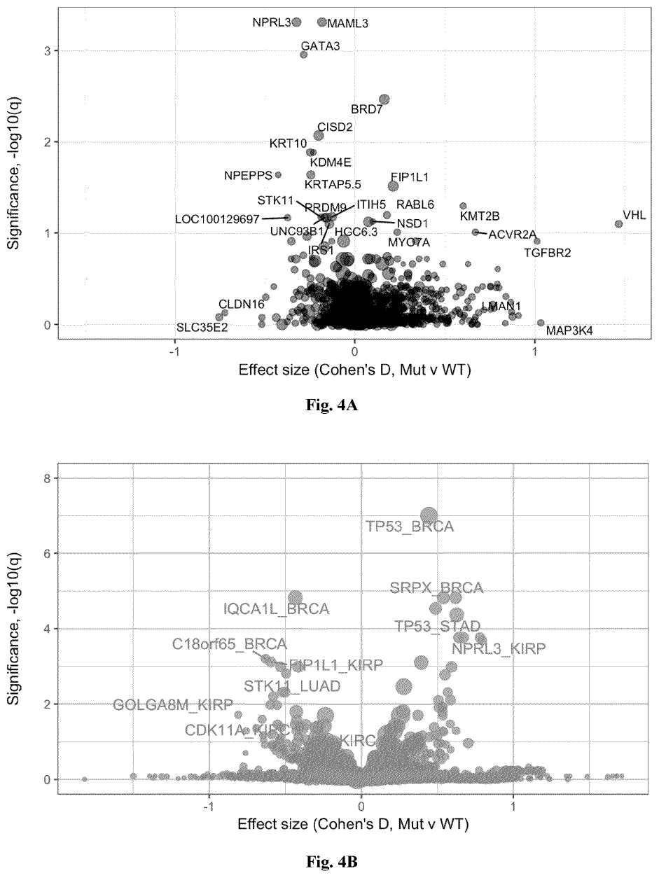

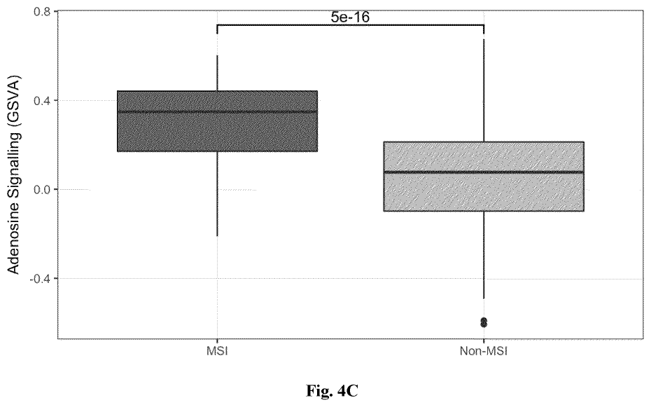

Adenosine signalling is not correlated with TMB at a pan-cancer level (r²=0.02), however MSI high tumours have significantly higher levels of adenosine signalling (FIG. 4C, p=5e⁻¹⁶). We therefore derived a linear model that incorporated MSI as a covariate with which to identify single nucleotide variants (SNVs) associated with adenosine signalling. Our analysis identifies 23 mutated genes that associate with adenosine signalling (at q<0.1) when all samples are considered in a pan-cancer model; 9 with enhanced adenosine signalling and 14 with reduced adenosine signalling (FIG. 4A and table 2).

6 adenosine associated genes have an established role in cancer pathogenesis, being members of the cancer gene census (39,40), including VHL, ACVR2A, FIP1L1 & NSD1 which all correlate with increased adenosine signalling, and GATA3 & STK11 that associate with reduced adenosine signalling (supplemental FIG. 3).

VHL has the largest effect size and is thought to be an E3 ubiquitin ligase that suppresses HIF1a expression. Thus VHL loss of function mutations lead to constitutive expression of HIF1a which upregulates CD73 and CD39, thereby enhancing the production of adenosine (41). This previously described mechanism gives further confidence in the relevance of the signature.

GATA3 is an important transcription factor associated with breast cancer and as a key regulator of CD4⁺ T cell development with some evidence to suggest its activity is regulated by adenosine in other settings (42).

The tumour suppressor STK11 has recently been shown to drive primary resistance to checkpoint inhibition (43) and the negative association with adenosine signalling identified here most likely reflects the immunologically cold/excluded tumour microenvironment for which an immuno-suppressive phenotype has not been activated. This raises the interesting possibility that the other negatively associated genetic segments might also exhibit resistance to immunotherapy. Notably, the most significantly associated genetic mutations are in NPRL3 which is part of the GATOR1 complex, which, like LKB1 via AMPK, feeds into the mTOR signalling pathway (44,45)

We found 55 SNVs associated with adenosine within an individual tumour type (q<0.05, FIG. 4B and supplemental table 2), comprising 25 from kidney renal papillary cell carcinoma, 23 from breast cancer, 3 from kidney renal clear cell carcinoma and 1 each from lung adenocarcinoma (STK11), prostate adenocarcinoma (RABL6), stomach adenocarcinoma (TP53) and head and neck squamous cell carcinoma (BRD7). 7 of these associations feature cancer census genes; TP53 in BRCA and STAR, GATA3 in BRCA, CDH1 in BRCA, VHL in KIRC, FIP1L1 in KIRP, STK11 in LUAD (supplemental FIG. 4).

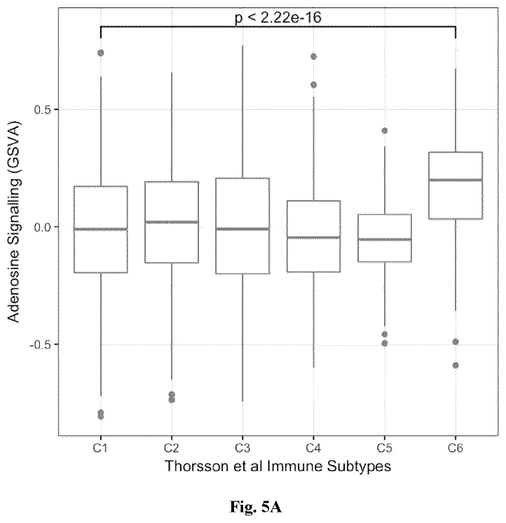

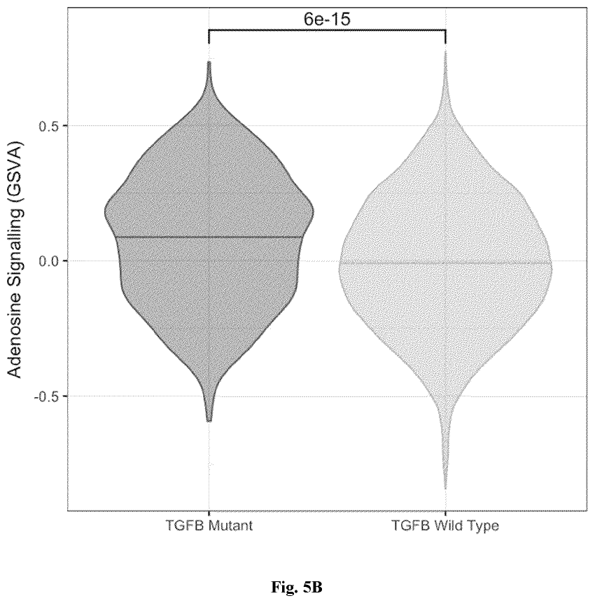

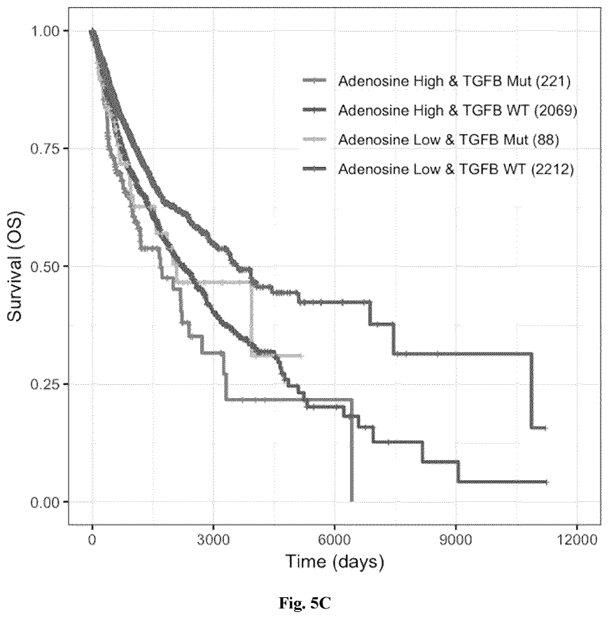

Somatic copy number alterations (SCNA) are also associated with adenosine signalling. 124 SCNA are significant (q<0.05) with 11 having an effect size greater than 0.5 (table 1 & supplemental FIG. 5). This includes a deletion on chromosome 3 which removes VHL and replicates the observation seen with SNVs.

TABLE 1
copy number variants associated with adenosine signalling with Cohen's D effect size > 0.5

| Location | Effect size | q | Type | Census Genes in Locus |
|---|---|---|---|---|
| chr3 32098168:37495009 | 1.54 | 0.0017 | DEL | CCR4, MLH1 |
| chr3 1:17201156 | 1.42 | 0.0370 | DEL | FANCD2, FBLN2, PPARG, |
| | | | | RAF1, SRGAP3, VHL, XPC |
| chr6 119669222:171115067 | 0.83 | 0.0413 | DEL | ARID1B, BCLAF1, ECT2L, |
| | | | | ESR1, EZR, FGFR1OP, |
| | | | | LATS1, MLLT4, MYB, PTPRK, |
| | | | | QKI, RSPO3, SGK1, TNFAIP3 |
| chr19 39363864:39953130 | 0.57 | 0.0096 | AMP | none |
| chr3 12384543:12494277 | 0.56 | 0.0000001 | AMP | none |
| chr19 30036025:30321189 | 0.54 | 0.0104 | AMP | CCNE1 |
| chr19 30183172:30321189 | 0.54 | 0.0104 | AMP | none |
| chr1 1:29140747 | 0.51 | 0.0012 | DEL | ARHGEF10L, ARID1A, CAMTA1, |
| | | | | CASP9, ID3, MDS2, MTOR, |
| | | | | PAX7, PRDM16, PRDM2, RPL22, |
| | | | | SDHB, SKI, SPEN, TNFRSF14 |
| chr1 150637495:150740723 | −0.89 | 0.0104 | AMP | none |
| chr1 228801039:249250621 | −0.90 | 0.0099 | AMP | AKT3, FH, RGS7 |
| chr8 113630879:139984811 | −0.94 | 0.0017 | AMP | CSMD3, EXT1, FAM135B, |
| | | | | MYC, NDRG1, RAD21 |

TABLE 2
23 genes harbouring SNVs associated with adenosine signalling (q < 0.1):

| | | | | | Cohen's | | | Is gene |
|---|---|---|---|---|---|---|---|---|
| | | | mut | | D effect | | | in |
| Gene | # mut | # wt | avg | wt avg | size | p | q | census? |
| MAML3 | 349 | 5525 | −0.043 | 0.003 | −0.183 | 5.57E−07 | 4.90E−04 | no |
| NPRL3 | 353 | 5521 | −0.077 | 0.005 | −0.324 | 9.64E−07 | 4.90E−04 | no |
| GATA3 | 154 | 5720 | −0.070 | 0.002 | −0.285 | 3.29E−06 | 1.11E−03 | yes |
| BRD7 | 518 | 5356 | 0.038 | −0.003 | 0.164 | 1.36E−05 | 3.44E−03 | no |
| CISD2 | 513 | 5361 | −0.046 | 0.005 | −0.202 | 4.22E−05 | 8.57E−03 | no |
| KDM4E | 90 | 5784 | −0.057 | 0.001 | −0.230 | 9.01E−05 | 1.31E−02 | no |
| KRT10 | 201 | 5673 | −0.061 | 0.003 | −0.249 | 8.60E−05 | 1.31E−02 | no |
| KRTAP5.5 | 295 | 5579 | −0.059 | 0.003 | −0.245 | 2.04E−04 | 2.30E−02 | no |
| NPEPPS | 85 | 5789 | −0.106 | 0.002 | −0.426 | 2.01E−04 | 2.30E−02 | no |
| FIP1L1 | 568 | 5306 | 0.049 | −0.005 | 0.213 | 3.02E−04 | 3.07E−02 | yes |
| KMT2B | 116 | 5758 | 0.150 | −0.003 | 0.602 | 5.47E−04 | 5.06E−02 | no |
| RABL6 | 179 | 5695 | 0.044 | −0.001 | 0.178 | 7.50E−04 | 6.35E−02 | no |
| ITIH5 | 356 | 5518 | −0.030 | 0.002 | −0.127 | 8.68E−04 | 6.66E−02 | no |
| STK11 | 88 | 5786 | −0.047 | 0.001 | −0.189 | 9.18E−04 | 6.66E−02 | yes |
| LOC100129697 | 128 | 5746 | −0.093 | 0.002 | −0.374 | 1.07E−03 | 6.80E−02 | no |
| PRDM9 | 458 | 5416 | −0.037 | 0.003 | −0.158 | 1.14E−03 | 6.80E−02 | no |
| UNC93B1 | 258 | 5616 | −0.041 | 0.002 | −0.169 | 1.09E−03 | 6.80E−02 | no |
| NSD1 | 73 | 5801 | 0.025 | 0.000 | 0.100 | 1.33E−03 | 7.49E−02 | yes |
| HGC6.3 | 504 | 5370 | 0.018 | −0.001 | 0.075 | 1.41E−03 | 7.52E−02 | no |
| IRS1 | 357 | 5517 | −0.034 | 0.003 | −0.142 | 1.65E−03 | 7.96E−02 | no |
| VHL | 162 | 5712 | 0.353 | −0.010 | 1.467 | 1.61E−03 | 7.96E−02 | yes |
| ACVR2A | 137 | 5737 | 0.166 | −0.004 | 0.670 | 2.14E−03 | 9.80E−02 | yes |
| MYO7A | 132 | 5742 | 0.059 | −0.001 | 0.235 | 2.22E−03 | 9.80E−02 | no |

### Example 5: Adenosine Signalling is Associated with TGF-β

TGFBR2 and ACVR2A mutations are amongst the most significant associations with adenosine levels in a pan-cancer model even after correction for MSI status. Both are members of the TGF-β superfamily encoding the TGF-β receptor and the structurally related activin growth factor receptor, respectively. TGF-β signalling has a complex and highly context dependent association with cancer biology. As a tumour suppressor, TGF-β mutation promotes tumourigenesis but its loss has also been shown to increase chemokine signalling resulting in infiltration of myeloid derived suppressor cells which themselves produce TGF-β and eventually drive immunosuppression thereby promoting tumour growth (46). Our result raises the possibility that this suppression is driven largely through the adenosine axis.

To further explore this relationship we conducted a deeper study of the association between adenosine and TGF-0. Thorsson et al (47) defined six primary immune subtypes of cancer including a TGF-β dominant group, cluster 6 (“C6”). We find that adenosine signalling is significantly higher in this group compared to the other five immune subtypes (FIG. 5A). We further expanded our analysis to include the 43 members of the TGF-β superfamily (48) and find that mutations in any of these genes are associated with a higher level of adenosine signalling (FIG. 5B). Finally, tumours that are both adenosine high and mutant in a TGF-β superfamily member have worse overall survival compared to tumours that are adenosine low and TGF-β wildtype (HR=0.43, p<2.2e⁻¹⁶), or those that are either TGF-β mutant (HR=0.74) or adenosine high (HR=0.72) (FIG. 5C).

### Example 6: Adenosine Signalling is Prognostic for Immunotherapy Response

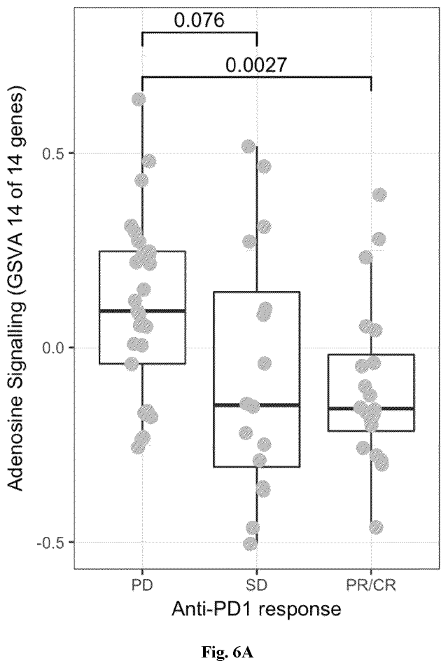

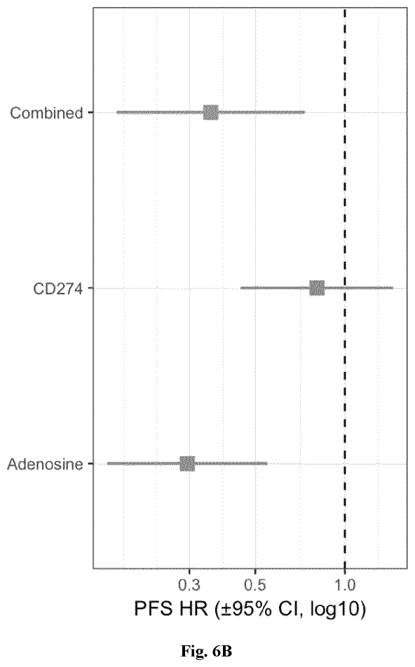

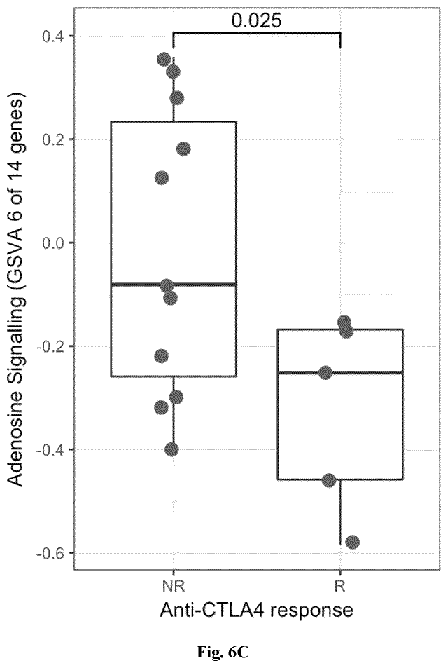

To test the clinical utility of the signature and the extent to which adenosine affects immune checkpoint therapy, we studied cohorts of patients treated with checkpoint inhibitors. Prat et al generated gene expression profiles of 65 patients from a variety of solid tumours that were treated with anti-PD1 therapy (49). Chen et al profiled 53 metastatic melanoma patients that were treated with anti-CTLA4 therapy (50). We find that responders to immune checkpoint therapy, as classified by their best overall response, have lower levels of baseline tumour adenosine signalling than do patients which progress on both anti-PD1 therapy (FIG. 6A) and anti-CTLA4 therapy (FIG. 6C). We used logistic regression to model the probability of a patient being a responder (CR, PR, SD) versus a non-responder (PD) in these cohorts. A signature score just below 0 (−0.01368) equates to a 50% probability of being a responder, and a signature score of −0.4 equates to a 75% probability of being a responder (supplemental figure S7).

In the anti-CTLA4 dataset only 6 genes from our 14 gene signature are present on the panel used. To study the effect this might have we scored the anti-PD1 dataset with the same 6 genes. The overall trend of results is retained but the sensitivity of the signature is reduced (PD v SD 6 gene p=0.072 versus 14 gene p=0.076, and PD v PR/CR 6 gene p=0.13 versus 14 gene p=0.0027).

There is also a highly significant association between adenosine signalling at baseline and progression free survival on anti-PD1 therapy (FIG. 6B; HR=0.29, Cox PH p=0.00012). Interestingly, expression of the gene encoding PDL1 (CD274), which is highly correlated with PDL1 IHC measurements (49), does not associate with progression free survival in the same dataset (HR=0.8, Cox PH p=0.47). Furthermore, combining adenosine and CD274 expression does not enhance the ability to predict immunotherapy response beyond adenosine alone (FIG. 6B and supplemental FIG. 6). These results would suggest that baseline levels of adenosine are an important determinant of response to immunotherapy and that our signature might complement PDL1 as a marker in this regard for the existing checkpoint inhibitors and potentially as A2AR inhibitors progress through the clinic.

### Example 7: Gene Expression Signatures for Adenosine-Drive Prostate Cancers

To derive signatures geared toward adenosine-drive prostate cancers, time-series of samples from 96 patients prostate cancer patients enrolled in the phase 1A study of AZD4635 (NCT02740985) were collected and analyzed for gene expression by using the NanoString nCounter FLEX Analysis System and the commercially available 770-gene, human PanCancer Immune Profiling Panel (NanoString). The time series was up to 120 weeks for some patients.

Signatures were tested for their concordance with progression-free survival (PFS), where progression is objective disease progression (by PSA recurrence or RECIST 1.1 criteria), or death by any cause in the absence of progression. (A concordance index of 0.5 represents random chance; a concordance index of 1 represents perfect prediction.) The concordance index, in this context, is a measure of how successful a given signature is in predicting PFS; that is, if stratifying patients into ‘adenosine-low’ and ‘adenosine-high’ groups correlated with the adenosine-high group having a longer median PFS than the adenosine-low group (patients with tumors that are more strongly adenosine drive should benefit more from treatment with the A2AR antagonist AZD4635). The stronger that correlation, the greater the concordance index.

The 14-gene signature described in Example 1 (group B) gave a concordance index of 0.584 when tested with the clinical prostate cancer data set.

Next, the group B signature was modified by adding one gene at a time from the 770 in the NanoString Immune Profiling Panel. Of the resulting 15-gene signatures, those with improved concordance indices were progressed, and the process of adding one gene to the signature and calculating the concordance index was repeated. Variations were also tested by omitting genes with low levels of expression, and with different numbers of genes in the signature (between 6 and 20). Results for illustrative signatures are shown in Table 3.

TABLE 3

| | Group | Group | Group | Group | Group | Group |
|---|---|---|---|---|---|---|
| | B | D | E | G | H | I |
| C-Index | 0.58 | 0.48 | 0.59 | 0.61 | 0.72 | 0.63 |

Group B: PPARG, PTGS2, FOXP3, COL3A1, MAPK3, LAG3, CD81, APP, MAPK1, FOS, CYBB, CREB1, GPI, CASP1
Group D: PTGS2, MAPK3, APP, MAPK1, FOS, GPI
Group E: PTGS2, MAPK3, LAG3, CD81, APP, MAPK1, FOS, CYBB, CREB1, GPI, CASP1
Group G: PPARG, COL3A1, MAPK3, LAG3, CD81, APP, FOS, CYBB
Group H: PPARG, COL3A1, MAPK3, LAG3, CD81, APP, FOS, CYBB, CASP1, TREM2, MUC1, MASP2, SPA17, CCR5, CD55, IL17B, CCL23, TARP, EBI3
Group I: PTGS2, MAPK3, LAG3, CD81, APP, MAPK1, FOS, CYBB, CREB1, GPI, CASP1, CCR5, CD55, TARP

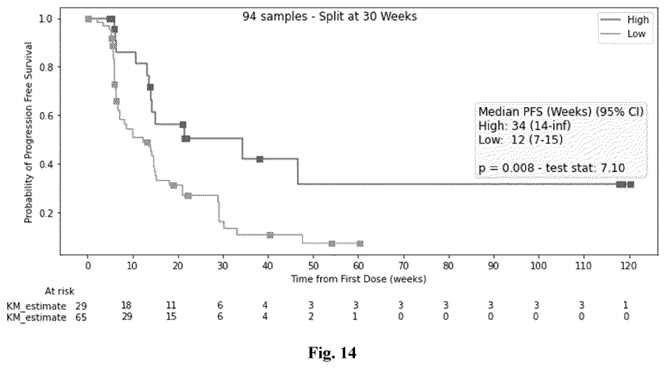

FIG. 14 is a Kaplan-Meier curve showing that for the signature of group I, the adenosine-high group showed a median PFS of 34 weeks, and the adenosine-low group 12 weeks (p=0.008).

What is claimed is:

1. A method for treating an adenosine-driven cancer in a subject, the method comprising:
diagnosing the subject with an adenosine-driven cancer when, in a sample from the subject, a signature score of tumour adenosine signalling is greater than a predetermined cutoff value;
wherein the signature score reflects the expression levels of a signature group of genes, wherein the signature group of genes includes at least three genes selected from group A: PPARG, CYBB, COL3A1, FOXP3, LAG3, APP, CD81, GPI, PTGS2, CASP1, FOS, MAPK1, MAPK3, CREB1, AKT3, TREM2, MUC1, CD164, FADD, FCGR2B, MASP2, ADA, SPA17, CCR5, CD55, IL17B, CD47, CCR2, CCL23, TARP, and EBI3; and
administering an effective amount of an adenosine signalling inhibitor to the diagnosed subject.

2. The method of claim 1, wherein the signature score is the GSVA score, mean, median, mode, or other statistical measure of the expression levels of the signature group of genes; and the signature score is optionally corrected for purity of the sample from the subject.

3. The method of any one of claim 1 or 2, wherein the predetermined cutoff value is the median, mean, top quartile, top quintile, top decile, or other statistical measure, of the signature score in a selected group of reference samples, and wherein the signature score is optionally corrected for sample purity within the selected group of reference samples.

4. The method of claim 3, wherein the selected group of reference samples includes a group of samples described in the Cancer Genome Atlas or a subset thereof.

5. The method of any one of claims 1 to 4, wherein the signature score is the GSVA score of the expression levels of the signature group of genes;
wherein the predetermined cutoff value is the median GSVA score of the expression levels of the signature group of genes in a selected group of reference samples;
wherein the selected group of reference samples includes a group of samples described in the Cancer Genome Atlas; and
wherein the signature score for the selected group of reference samples is corrected for sample purity.

6. The method of claim 5, wherein the signature group of genes includes:
at least five genes selected from group A;
at least five genes selected from group B;
at least three genes selected from group C;
at least five genes selected from group D;
at least five genes selected from group E;
at least five genes selected from group F;
at least five genes selected from group G;
at least five genes selected from group H; or
at least five genes selected from group I.

7. The method of claim 5, wherein the signature group of genes is:
group A;
group B;
group C;
group D;
group E;
group F;
group G;
group H; or
group I.

8. The method of any one of claims 1 to 7, wherein the signature group of genes includes MAPK3, LAGS, CD81, APP, FOS, and CYBB.

9. The method of any one of claims 1 to 8, wherein diagnosing the subject further comprises determining that: the cancer has a mutation in one or more genes selected from VHL, ACVR2A, FIP1L1, NSD1, GATA3, or STK11.

10. The method of any one of claims 1 to 9, wherein diagnosing the subject further comprises determining that: the cancer has an SNV in one or more genes selected from MAML3, NPRL3, GATA3, BRD7, CISD2, KDM4E, KRT10, KRTAP5.5, NPEPPS, FIP1L1, KMT2B, RABL6, ITIH5, STK11, LOC100129697, PRDM9, UNC93B1, NSD1, HGC6.3, IRS1, VHL, ACVR2A, and MYO7A.

11. The method of any one of claims 1 to 10, wherein diagnosing the subject further comprises determining that: the cancer has a somatic copy number alteration (SCNA) at one or more locations selected from:
chr3 32098168:37495009,
chr3 1:17201156,
chr6 119669222:171115067,
chr19 39363864:39953130,
chr3 12384543:12494277,
chr19 30036025:30321189,
chr19 30183172:30321189,
chr1 1:29140747,
chr1 150637495:150740723,
chr1 228801039:249250621, and
chr8 113630879:139984811.

12. The method of any one of claims 1 to 11, wherein diagnosing the subject further comprises determining that the cancer has a mutation in a gene belonging to the TGF-β superfamily.

13. The method of any one of claims 1 to 12, wherein the cancer is prostate cancer, breast cancer, colon cancer, lung cancer, uveal melanoma, cervical cancer, pancreatic cancer, or thyroid cancer.

14. The method of any one of claims 1 to 13, wherein the cancer is prostate cancer.

15. The method of claim 14, wherein the signature group of genes includes:
at least five genes selected from group E;
at least five genes selected from group F;
at least five genes selected from group G;
at least five genes selected from group H; or
at least five genes selected from group I.

16. The method of claim 15 wherein the signature group of genes includes at least five genes selected from group I.

17. The method of claim 16, wherein the signature group of genes is group I.

18. The method of any one of claims 1 to 17, wherein the adenosine signalling inhibitor includes a CD39 inhibitor, a CD73 inhibitor, an adenosine receptor antagonist, or a combination thereof.

19. The method of any one of claims 1 to 18, wherein the adenosine signalling inhibitor is IPH5201, oleclumab, AZD4635, or a combination thereof.

20. The method of any one of claims 1 to 19, further comprising administering an effective amount of an immune checkpoint inhibitor to the diagnosed subject.

21. The method of claim 20, wherein the immune checkpoint inhibitor is durvalumab, atezolizumab, avelumab, nivolumab, pembrolizumab, cemiplimab, tremelimumab, or ipilimumab.

22. Use of an adenosine signalling inhibitor for the treatment of an adenosine-driven cancer in a subject, wherein:
in a sample from the subject, a signature score of tumour adenosine signalling is greater than a predetermined cutoff value;
wherein the signature score reflects the expression levels of a signature group of genes, wherein the signature group of genes includes at least three genes selected from
at least five genes selected from group A;
at least five genes selected from group B;
at least three genes selected from group C;
at least five genes selected from group D;
at least five genes selected from group E;
at least five genes selected from group F;
at least five genes selected from group G;
at least five genes selected from group H; or
at least five genes selected from group I.

23. The use of claim 24, wherein the signature group of genes is:
group A;
group B;
group C;
group D;
group E;
group F;
group G;
group H; or
group I.

25. The use of any one of claims 22 to 24, wherein the cancer is prostate cancer, breast cancer, colon cancer, lung cancer, uveal melanoma, cervical cancer, pancreatic cancer, or thyroid cancer.

26. The use of any one of claims 22 to 25, wherein the cancer is prostate cancer.

27. The use of claim 26, wherein the signature group of genes includes:
at least five genes selected from group E;
at least five genes selected from group F;
at least five genes selected from group G;
at least five genes selected from group H; or
at least five genes selected from group I.

28. The use of claim 27, wherein the signature group of genes includes at least five genes selected from group I.

29. The use of claim 28, wherein the signature group of genes is group I.
